(19) United States
(12) Patent Application Publication
(10) Pub. No.: US 2023/0329650 A1
(43) Pub. Date: Oct. 19, 2023

(54) MEDICAL MONITORING SYSTEMS AND METHODS

(71) Applicant: ABBOTT DIABETES CARE INC., Alameda, CA (US)

(72) Inventors: Aparajita Bhattacharya, Dublin, CA (US); Gary A. Hayter, Oakland, CA (US)

(21) Appl. No.: 18/019,289

(22) PCT Filed: Aug. 4, 2021
(86) PCT No.: PCT/US21/44462

§ 371 Date: Feb. 2, 2023

Related U.S. Application Data
(60) Provisional application No. 63/061,704, filed on Aug. 5, 2020.

(51) Int. Cl. A61B 5/00; G16H 50/70; G16H 15/00

(52) U.S. Cl. CPC A61B 5/7475; G16H 50/70; G16H 15/00; G16H 40/67

(57) ABSTRACT

An interactive graphical user interface associates information configured for interactive display and input, including patient identifying information for each patient in a patient list, a medication schedule for the each patient, and a treatment assessment worksheet comprising a display indicating the medical monitoring data for the each patient. The worksheet enables comparing monitoring results over different periods of time and development of treatment plans.

### CROSS-REFERENCE TO RELATED APPLICATIONS

This application is a national phase entry under 35 U.S.C. § 371 of PCT Application No. PCT/US21/44462, filed Aug. 4, 2021, which claims priority to U.S. Application Ser. No. 63/061,704, filed Aug. 5, 2020, both of which are hereby expressly incorporated by reference in their entireties for all purposes.

### FIELD

The present application relates to an electronic interface, such as a graphical user interface for medical monitoring and management of treatment, as well as systems, devices, and methods relating thereto.

### BACKGROUND

Various wearable monitoring devices exist for interstitial or intravenous monitoring of an analyte indicative of a health condition. For example, in the field of diabetes treatment, sensor control devices are available that monitor glucose in interstitial tissue, using a sensor that is implanted under the skin and coupled to a processor, memory, power supply and wireless interface contained inside a housing that is adhered to the patient's skin. The processor takes periodic sensor reading, stores the sensor data in the memory, and may communicate sensor data to another device, for example, a smartphone of the patient, or a notepad computer, personal computer, or similar processing and display device of a medical practitioner. In some cases, the smartphone or other device is configured to upload monitoring data to a remote server for safekeeping and distribution to authorized medical practitioners. Thus, a wealth of monitoring data obtained at intervals over continuous periods can be made available to the patient and medical practitioner for use in managing treatment of health conditions, including but not limited to diabetes.

However, in some circumstances it may be difficult for a health practitioner to make optimal use of monitoring data from wearable monitoring devices. While electronic interfaces, including graphical user interfaces, are known and used for accessing monitoring information, often the medical practitioner needs supplemental information, guidance in interpreting monitoring data, or guidance in treatment recommendations to make the most effective use of available data. Presently, electronic interfaces for use by medical practitioners in accessing and applying medical monitoring data from wearable devices lack helpful features, or do not arrange the features in an optimal way for workflow.

It would be desirable, therefore, to develop new methods and other new technologies for electronic interfaces, such as graphical user interfaces for medical monitoring and management of treatment, that overcomes these and other limitations of the prior art.

### SUMMARY

This summary and the following detailed description should be interpreted as complementary parts of an integrated disclosure, which parts may include redundant subject matter and/or supplemental subject matter. An omission in either section does not indicate priority or relative importance of any element described in the integrated application. Differences between the sections may include supplemental disclosures of alternative embodiments, additional details, or alternative descriptions of identical embodiments using different terminology, as should be apparent from the respective disclosures.

In an aspect of the disclosure, a method for an electronic interface of a computing device, may include detecting, by at least one processor, an identifier for a sensor control device worn by a patient within wireless range of a receiver coupled to the at least one processor. The method may further include receiving, by the at least one processor, medical monitoring data collected by the sensor control device over a continuous period. The method may include providing, to a display device, an interactive graphical user interface associating information configured for interactive display and input, the information comprising patient identifying information for each patient in a patient list, a medication schedule for the each patient, and a treatment assessment worksheet comprising a display indicating the medical monitoring data for the each patient. In some embodiments, the method may also include providing, to a display device, an interactive graphical user interface associating information configured for interactive display and input, the information comprising an intervention screen configured to allow a provider to indicate patterns (issues) that the provider will be addressing during a current visit; edits, adds, and/or deletions to a patient's medications; and self-care actions that the patient can try to follow. The method may include receiving data input via the interactive graphical user interface for each patient during a patient visit, and storing the data input in a record for each patient. The method may include further details and operations as described in the detailed disclosure that follows.

The method may be implemented by any suitable user interface device that can receive medical monitoring data for an identified patient. As used herein, a “user interface device” includes at least a computer processor coupled to a memory and to one or more ports, including at least one input port and at least one output port (e.g., a desktop computer, laptop computer, tablet computer, smartphone, PDA, etc.). A computer processor may include, for example, a microprocessor, microcontroller, system on a chip, or other processing circuit. As used herein, a “processor” means a computer processor.

To the accomplishment of the foregoing and related ends, one or more examples comprise the features hereinafter fully described and particularly pointed out in the claims. The following description and the annexed drawings set forth in detail certain illustrative aspects and are indicative of but a few of the various ways in which the principles of the examples may be employed. Other advantages and novel features will become apparent from the following detailed description when considered in conjunction with the drawings and the disclosed examples, which encompass all such aspects and their equivalents.

### BRIEF DESCRIPTION OF THE DRAWINGS

The features, nature, and advantages of the present disclosure will become more apparent from the detailed description set forth below when taken in conjunction with the drawings in which like reference characters identify like elements correspondingly throughout the specification and drawings.

FIG. 1 shows an overview of an analyte monitoring system comprising a sensor applicator, a sensor control device, a reader device, a network, a trusted computer system, and a local computer system.

FIG. 2A is a block diagram illustrating an example embodiment of a reader device.

FIGS. 2B and 2C are block diagrams illustrating example embodiments of sensor control devices.

FIG. 2D is a block diagram illustrating an example embodiment of a user interface device.

FIG. 3 is a flow diagram illustrating operations of a method for providing a graphical user interface for medical monitoring and management of treatment.

FIG. 4 is a flow diagram illustrating further, optional aspects of the method diagrammed in FIG. 3.

FIGS. 5A-5C are graphical user interfaces illustrating aspects of a patient encounters portion of the graphical user interface and method.

FIGS. 6A-6E are graphical user interfaces illustrating aspects of a patient information portion of the graphical user interface and method.

FIGS. 7A-7D are graphical user interfaces illustrating aspects of a visit information portion of the graphical user interface and method.

FIG. 8 is a flow diagram illustrating further, optional aspects of the method diagrammed in FIG. 3.

FIGS. 9A-9G are graphical user interfaces illustrating aspects of a medication portion of the graphical user interface and method.

FIG. 10 is a flow diagram illustrating further, optional treatment assessment aspects of the method diagrammed in FIG. 3.

FIGS. 11A-11D are graphical user interfaces illustrating aspects of a treatment assessment portion of the graphical user interface and method.

FIG. 12 is a flow diagram illustrating one or more additional operations for providing human-readable observations and recommendations in treatment assessment, that may be included in the method diagrammed in FIG. 3.

FIG. 13A is a table showing examples of assessment outcomes as may be managed and displayed using the graphical user interface and method.

FIG. 13B is a flow diagram illustrating aspects of a method by a processor for selecting text for expressing assessment outcomes.

FIG. 14 is a conceptual block diagram illustrating components of an apparatus or system for providing a graphical user interface for medical monitoring and management of treatment.

### DETAILED DESCRIPTION

Various aspects are now described with reference to the drawings. In the following description, for purposes of explanation, numerous specific details are set forth to provide a thorough understanding of one or more aspects. It may be evident, however, that the various aspects may be practiced without these specific details. In other instances, well-known structures and devices are represented in block diagram form to facilitate focus on novel aspects of the present disclosure. While the example embodiments related to monitoring for glucose concentration and related management of treatment for diabetes, the inventive concepts herein may be extended to graphical user interfaces for monitoring and treating other conditions or diseases.

Embodiments disclosed herein relate to improved GUIs or GUI features for analyte monitoring systems that are intuitive, user-friendly, and facilitate assessment of physiological information of a patient by a medical practitioner. For example, these embodiments allow a medical practitioner to rapidly and thoroughly assess medical monitoring data for the patient, review the patient's medication and self-care records synchronized to graphical displays of monitoring data, identify potential shortcomings in treatment, refine prescriptions for medications, and counsel the patient regarding self care. Aspects of the GUIs and GUI features, such as the guided interpretation reports, equip the medical practitioner to better understand the practical impact of medication, patient habits and response to medication, and refine the treatment for a disease or medical condition. Likewise, embodiments provided herein comprise improved digital interfaces and/or features for assessment of massive data collected by automated analyte monitoring systems for rapid and accurate assessment of the effect of past interventions, issues that recur at particular times-of-day, collect relevant data in a compact interface for rapid assimilation, organize patient records, streamline patient intake during clinical visits, and conveniently create accurate records of finding and interventions for the patient's medical records, among other benefits and advantages disclosed herein.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Generally, embodiments of the present disclosure include GUIs and digital interfaces for analyte monitoring systems, and methods and devices relating thereto. Accordingly, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for every embodiment of a method disclosed herein, systems and devices capable of performing each of those embodiments are covered within the scope of the present disclosure. For example, embodiments of sensor control devices, reader devices, local computer systems, and trusted computer systems are disclosed, and these devices and systems can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor may be an invasive sensor, for example a glucose sensor configured for insertion under the skin for sensing glucose in interstitial tissue, or a blood glucose sensor. In alternative embodiments, the sensor may sense a different analyte, or may be non-invasive. The sensor control device may be equipped to measure different quantities in addition to glucose or other analytes, for example body temperature, pulse, or blood oxygen level. The sensor control device, and variations thereof, can also be referred to as a “sensor control unit,” an “on-body electronics” device or unit, an “on-body” device or unit, or a “sensor data communication” device or unit, for example.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Example Embodiment of In Vivo Analyte Monitoring System

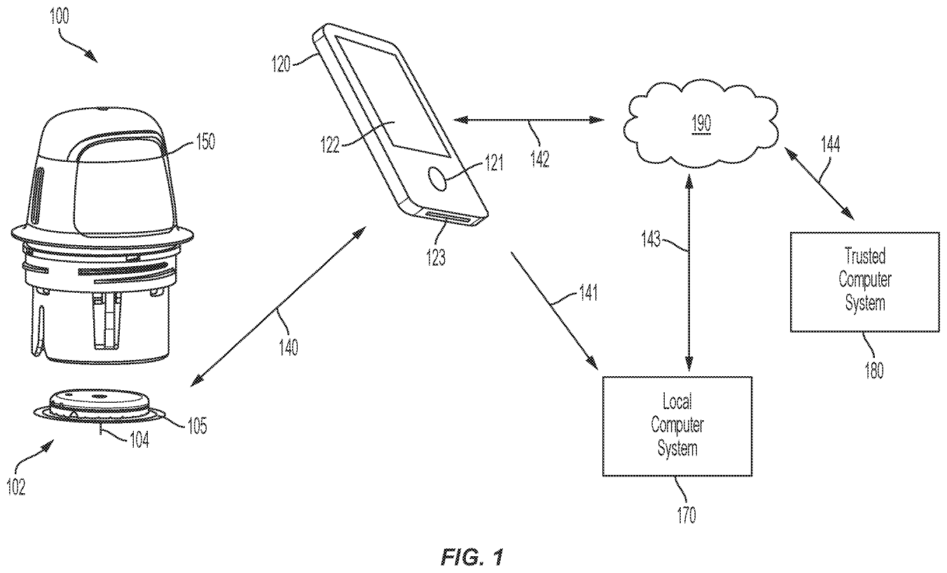

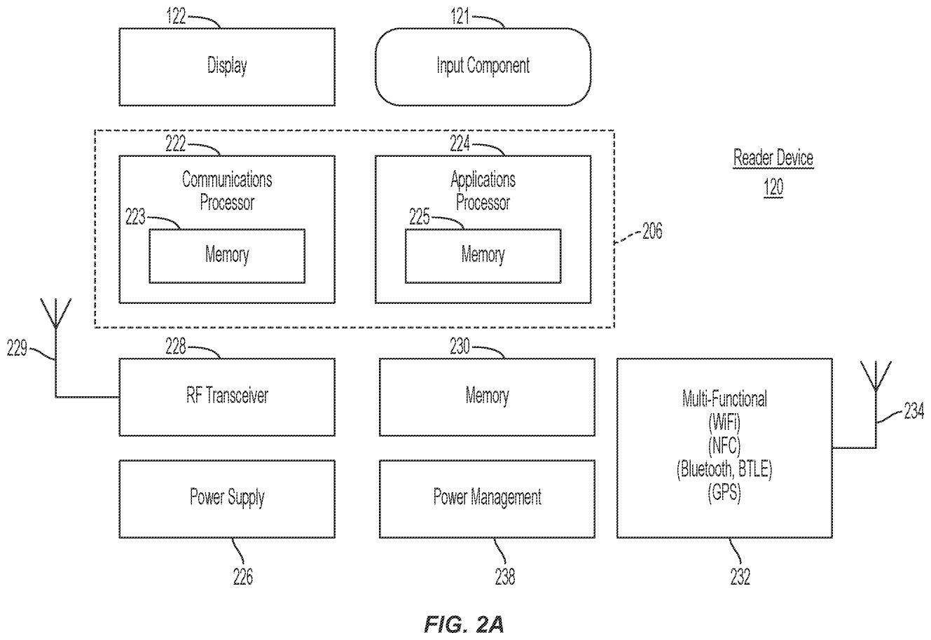

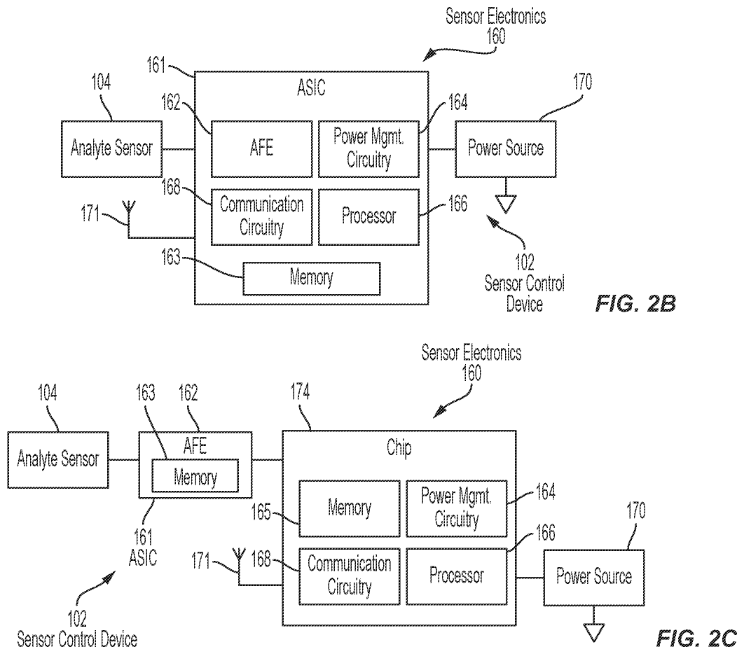

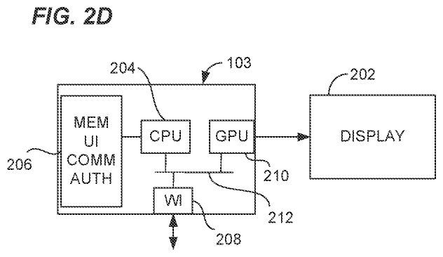

FIG. 1 is a conceptual diagram depicting an example embodiment of an analyte monitoring system 100 that includes a sensor applicator 150, a sensor control device 102, and a reader device 120. Here, sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can view and use applications installed in memory on reader device 120 using screen 122 (which, in many embodiments, can comprise a touchscreen), and input 121. A device battery of reader device 120 can be recharged using power port 123. While only one reader device 120 is shown, sensor control device 102 can communicate with multiple reader devices 120. Each of the reader devices 120 can communicate and share data with one another. More details about reader device 120 is set forth with respect to FIG. 2A below. Reader device 120 can communicate with local computer system 170 via a communication path 141 using a wired or wireless communication protocol.

Local computer system 170 may be, or may include, one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how reader device 120 can communicate via a communications path 142 with network 190, by a wired or wireless communication protocol as described previously. Network 190 can be any of several networks, such as private networks and public networks, local area or wide area networks, and so forth. In some embodiments, the local computer system 170 may function as a user interface device operating an interactive graphical user interface (GUI) as described herein, for example in a clinical setting for use by a medical practitioner. The local computer 170 may include components the same as, or equivalent to components of the reader device 120, in the same form factor or in a different form factor. For example, the reader device may by a smart phone and the local computer may be a laptop or personal computer.

A trusted computer system 180 may include a cloud-based platform or server, and can provide for authentication services, secured data storage, report generation, and can communicate via communications path 144 with network 190 by wired or wireless technique. In addition, although FIG. 1 depicts trusted computer system 180 and local computer system 170 communicating with a single sensor control device 102 and a single reader device 120, it will be appreciated by those of skill in the art that local computer system 170 and/or trusted computer system 180 are each capable of being in wired or wireless communication with a plurality of reader devices and sensor control devices.

### Example Embodiment of Reader Device

FIG. 2A is a block diagram depicting an example embodiment of a reader device 120, which, in some embodiments, can comprise a smart phone. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further, reader device 120 can also include a multi-functional transceiver 232, which can comprise wireless communication circuitry, and which can be configured to communicate over Wi-Fi, NFC, Bluetooth, BTLE, and GPS with an antenna 234. As understood by one of skill in the art, these components are electrically and communicatively coupled in a manner to make a functional device.

### Example Embodiments of Sensor Control Devices

FIGS. 2B and 2C are block diagrams depicting example embodiments of sensor control devices 102 having analyte sensors 104 and sensor electronics 160 (including analyte monitoring circuitry) that can have the majority of the processing capability for rendering end-result data suitable for display to the user. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed among two or more of them. Memory 163 can also be a separate chip. Memory 163 can be volatile and/or non-volatile memory. In this embodiment, ASIC 161 is coupled with power source 172, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data. According to some embodiments, for example, a current glucose value can be transmitted from sensor control device 102 to reader device 120 every minute, and historical glucose values can be transmitted from sensor control device 102 to reader device 120 every five minutes.

In some embodiments, to conserve power and processing resources on sensor control device 102, digital data received from AFE 162 can be sent to reader device 120 (not shown) with minimal or no processing. In still other embodiments, processor 166 can be configured to generate certain predetermined data types (e.g., current glucose value, historical glucose values) either for storage in memory 163 or transmission to reader device 120 (not shown), and to ascertain certain alarm conditions (e.g., sensor fault conditions), while other processing and alarm functions (e.g., high/low glucose threshold alarms) can be performed on reader device 120. Those of skill in the art will understand that the methods, functions, and interfaces described herein can be performed—in whole or in part—by processing circuitry on sensor control device 102, reader device 120, local computer system 170, or trusted computer system 180. As used herein, a “user interface device” is, or includes, one of these devices that controls an interactive graphical user interface appearing on a display device.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 may include memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### Example Embodiments of Graphical User Interfaces for Analyte Monitoring Systems

Described herein are example embodiments of GUIs for analyte monitoring systems. As an initial matter, it will be understood by those of skill in the art that the GUIs described herein comprise instructions stored in a memory of reader device 120, local computer system 170, trusted computer system 180, and/or any other device or system that is part of, or in communication with, analyte monitoring system 100. These instructions, when executed by one or more processors of the reader device 120, local computer system 170, trusted computer system 180, or other device or system of analyte monitoring system 100, cause the one or more processors to perform the method steps and/or output the GUIs described herein. Those of skill in the art will further recognize that the GUIs described herein can be stored as instructions in the memory of a single centralized device or, in the alternative, can be distributed across multiple discrete devices in geographically dispersed locations.

The processor of the sensor control device 102 may be configured to receive and process sensor data at periodic intervals, or in response to detected events, and store the measurements of analyte readings (e.g., glucose measurements) in a local memory. In addition, the processor may via its wireless interface communicate sensor data to a user interface device, e.g., reader device 120, local computer system 170, or trusted computer system 180, directly or via an intermediate node, for example, via one or more networks. In alternative embodiments, the sensor control device may transmit data to a local receiver, e.g., reader device 120 using a BLE or other suitable protocol, and the local receiver may forward the data to a remote computer. This is useful when the medical practitioner using the user interface device is located remotely from the patient. In other embodiments, for example, in certain clinical settings, the physician or other medical practitioner operating the user interface device may receive data directly from the sensor control device 102 via its wireless interface. In the examples considered herein, the monitoring data includes glucose measurements made at intervals by the sensor control device over a defined period or periods.

An application running on the user interface device may acquire real-world medication data and glucose data for the patient during a patient visit. The application is intended to be used by medical assistants (MA), physicians, or other qualified medical practitioners. For the sake of brevity, such persons are sometimes referred to herein as “users.” Patients may use any commercially available sensor control device that is compatible with the user interface application. The user interface application may cause the user interface device to display glucose data (e.g., an Ambulatory Glucose Profile, AGP) and enables data entry of medication data by the medical practitioner in association with the patient record. The interactive graphical user interface application may be designed to facilitate use of the AGP and medication records to assess the impact of past therapy changes and adjust treatment recommendations. After the patient visit is completed, the user interface application may generate a summary document for adding to an existing Electronic Medical Record (EMR) for the patient.

FIG. 2D shows further details of a user interface device 103, which may include at least one processor 204 coupled to a memory 207, and to a graphic processing unit (GPU) 210 and wireless interface 208 via a bus 212 or other suitable connection. The processor 204 may send display output and related instructions to the GPU 210, which generates a video signal for the display 202, for example, an LED monitor or touchscreen. When the display 202 is configured as a touchscreen, it may also serve as a data input device for receiving input from a person using the user interface displayed on the display device 202. In other embodiments, the processor 204 may be coupled to one or more additional input devices, for example, a keyboard, microphone, or pointing device, via a suitable interface.

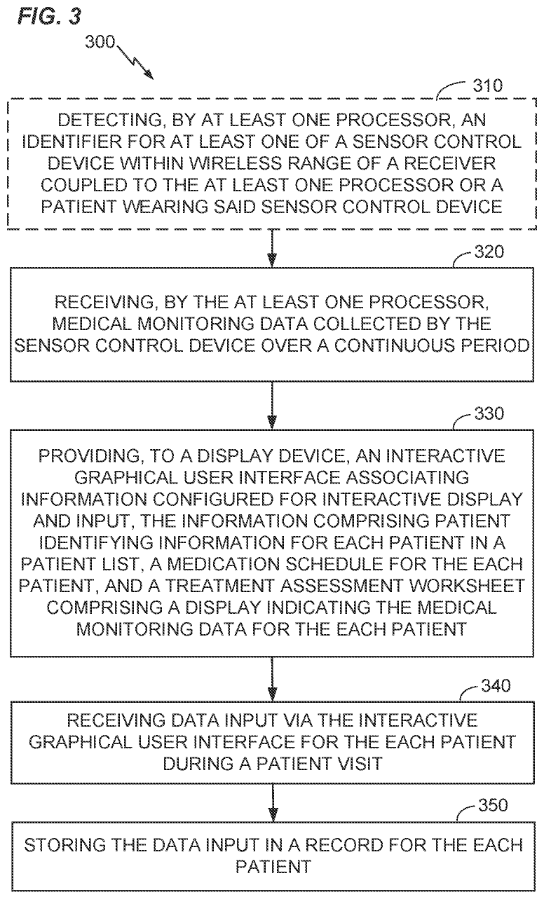

The memory 207 may include one or more code modules that when executed by the programmer produce an interactive graphical user interface as described herein. Any suitable programming language may be used to encode the modules, including but limited to a web application language such as JavaScript and/or PHP, or a compiled language such C++. Modules may include, for example, one or more user interface modules as described herein, and other modules, for example, communication and authentication modules as known in the art. One or more modules in the memory 207, when executed by the at least one processor 204, may cause the user interface device 103 to perform operations of the method 300 as diagrammed in FIG. 3, or a variation thereof.

Referring to FIG. 3, the computer-implemented method 300 for an electronic interface of a computing device (e.g., local computer system 170, reader device 120, or user interface device 103) may include, at 310 detecting, by at least one processor, an identifier for one or more of a patient identifier or a sensor control device worn by a patient within wireless range of a receiver coupled to the at least one processor. In an alternative, or in addition, the at least one processor may receive data from the sensor control device, and/or additional medical or patient information, through an intermediate node or server. It should be appreciated that the operation 310 need not be included in all embodiments. Instead, for example, the user may manually identify the patient by data entry. If automatic detection is used, the processor may receive the identifier from the sensor control device, and optionally, may link an identifier for the sensor control device to a patient identifier.

Thus, the method 300 may include, at 320, receiving, by the at least one processor, medical monitoring data collected by the sensor control device over a predetermined period. The method 300 may further include, at 330, providing an interactive graphical user interface to a display device. Examples of various states of such a graphical user interface are provided in the accompanying figures representing screenshot, or partial screenshot examples. The graphical user interface may associate information configured for interactive display and one or more fields for input by the user. The information may include patient identifying information for each patient in a patient list, a medication schedule for the each patient, and a treatment assessment worksheet comprising a display indicating the medical monitoring data for the each patient, and/or further information as described herein. The method 300 may include, at 340, receiving data input via the interactive graphical user interface for the each patient during a patient visit and, at 350, storing the data input in a record for the each patient. The foregoing operations are further illustrated and described in connection with the figures described below.

The method 300 may include any one or more additional novel operations as described herein, and other operations as known in the art. For example, the method may include an operation for authenticating the user before providing access to the interactive graphical interface. Each of these additional operations is not necessarily performed in every embodiment of the method, and the presence of any one of the operations does not necessarily require that any other of these additional operations also be performed.

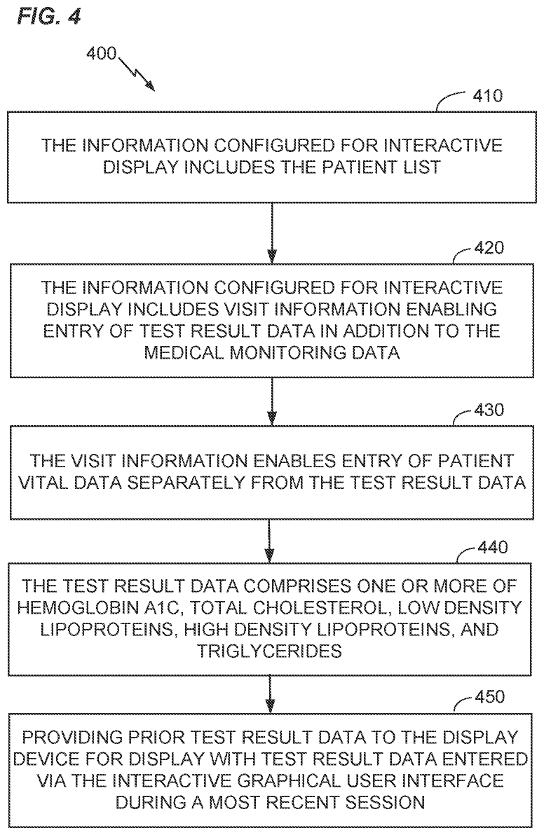

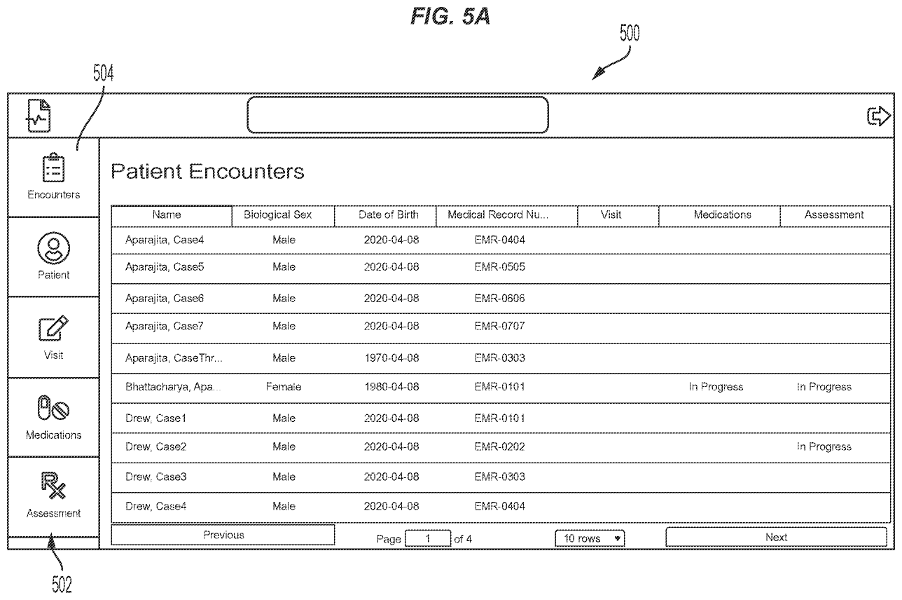

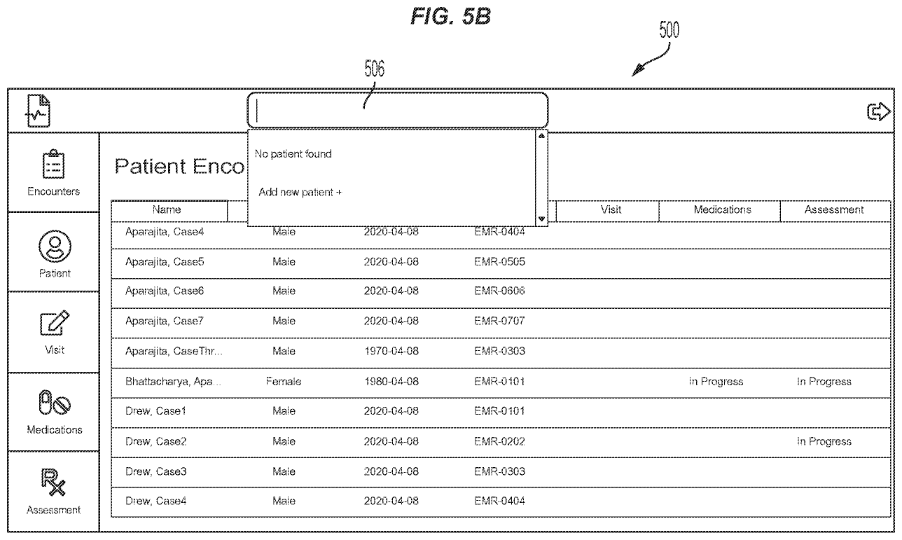

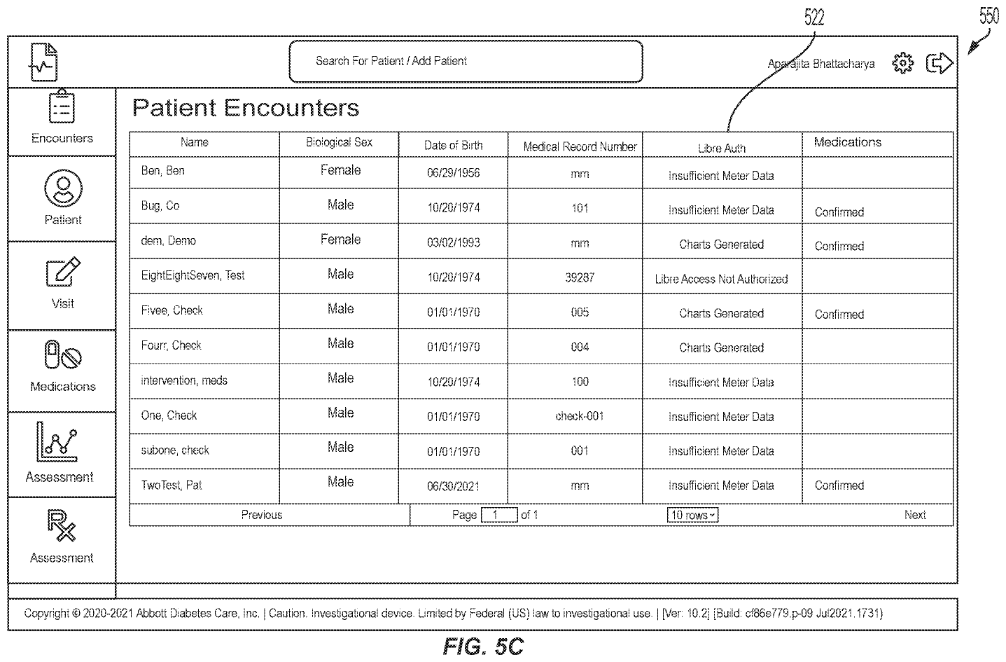

For example, FIG. 4 shows additional operations 400 for enhancing operation of the interactive graphical user interface. At 410, the method 300 may further include generating the information configured for interactive display including a patient list. FIGS. 5A and 5B show examples of a patient list in related screenshots 500. After the processor authenticates the user, it may generate a display like screenshots 500, here labeled “Encounters” as shown by highlighted tab 504 in the vertical tab list 502. The Encounters screen 500 includes the patient list, listing patients in the memory of the user interface device by reason of prior registration. In some embodiments, the processor causes patient data to appear on the Encounters screen 500 only if the visit is initiated and not yet completed. In another aspect shown in FIG. 5B, if the user types a character string into the search field 506, the processor may, using an index or other method, cause patient encounters matching the character screen to appear in the list. If the patient does not appear, the user may instruct the processor to add a new patient record, via the interactive graphical user interface.

In another aspect, the processor may cause certain columns in the patient list, e.g., the rightmost three columns in screen 500, to display status of the patient encounter. In the illustrated example, the status of the process tabs “Visit,” Medications,” and “Assessment,” which will be described in more detail below, are indicated in screen 500.

FIG. 5C depicts another example embodiment of an Encounters screen 550. In several aspects, the interface shown in screen 550 is similar to screen 500 described with respect to FIGS. 5A and 5B. According to some embodiments, screen 550 can further comprise a column 552, here labeled “Libre Auth,” to indicate a status of the glucose data for each row. By way of example, some values that can be displayed in column 552 can include “Libre Access Not Authorized” (to indicate that the system has not been authorized to retrieve, process, and/or display a patient's glucose data), “Retrieving/Processing Data” (to indicate that the system is in the process of retrieving and/or processing the glucose data), “Insufficient Data,” or “Charts Generated.” Those of skill in the art will recognize that other values to reflect the status of glucose data can be displayed and are fully within the scope of the present disclosure.

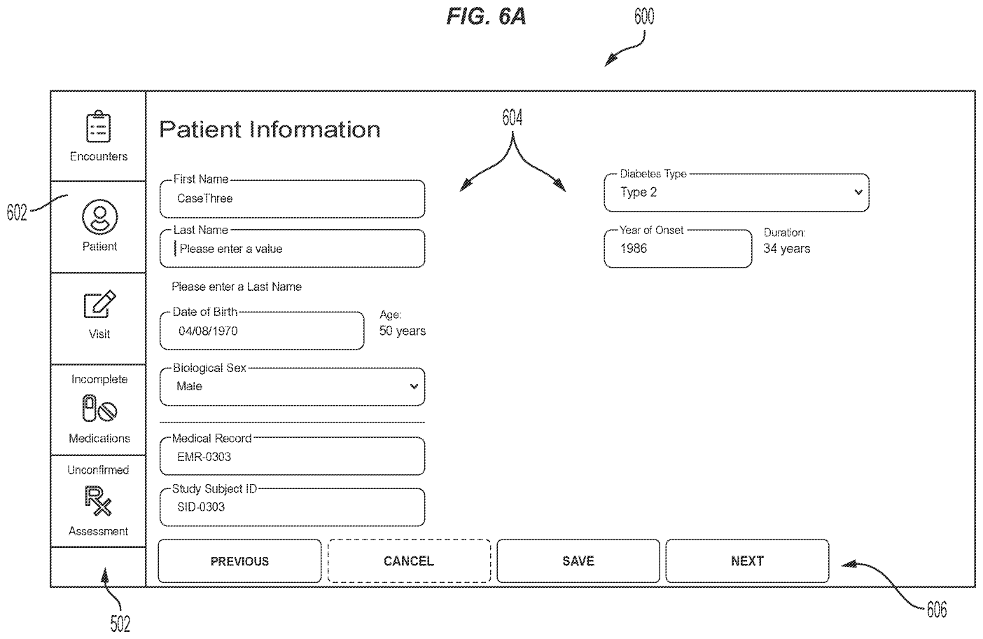

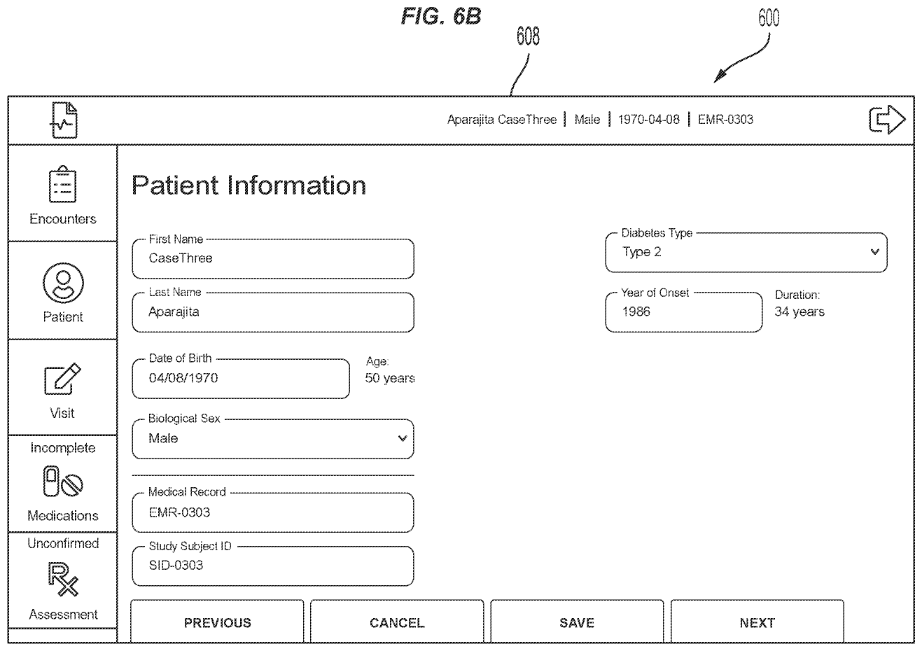

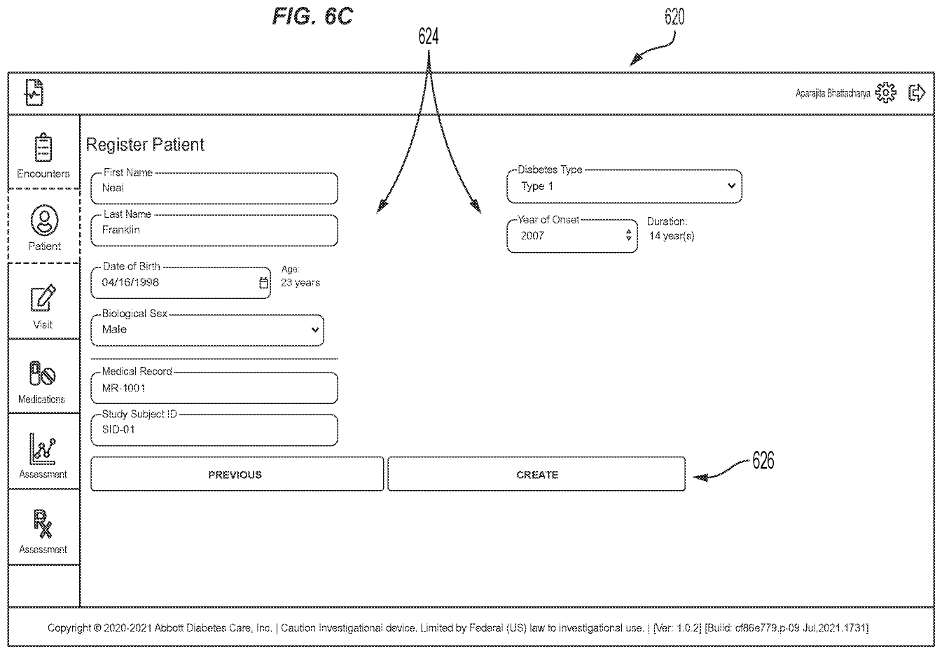

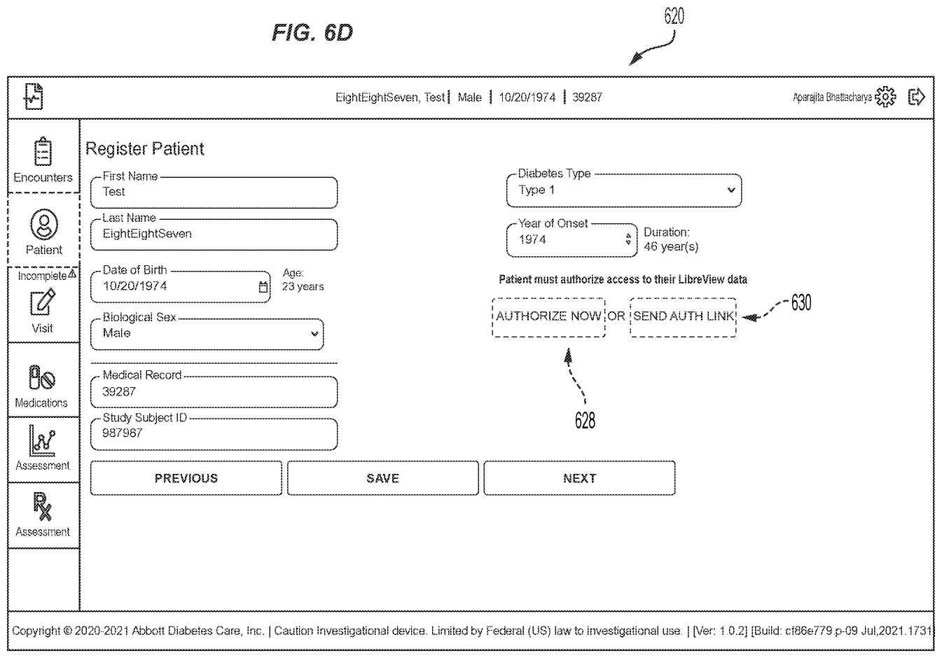

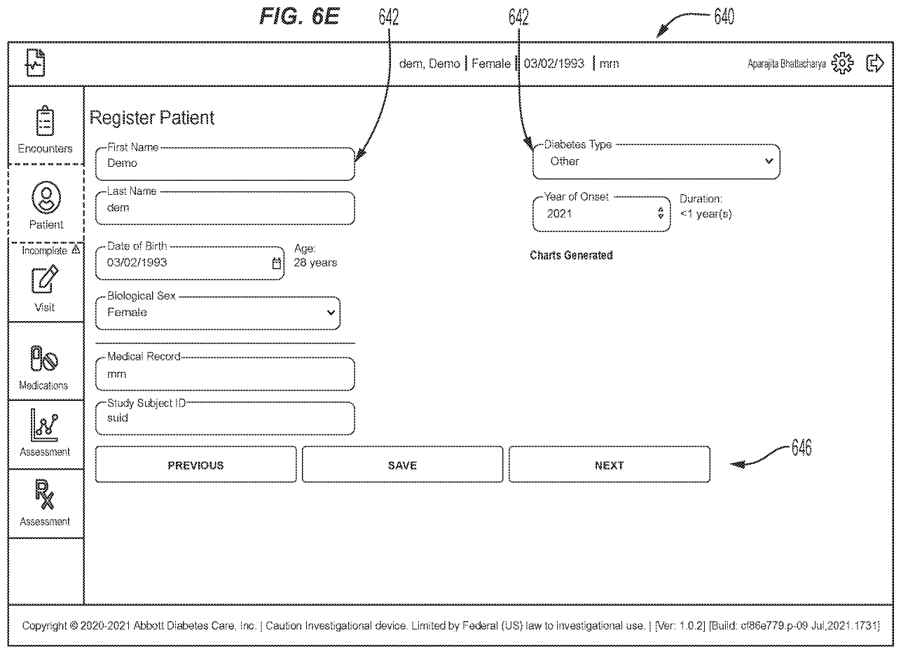

FIGS. 6A and 6B show examples of a patient information screen 600 under the information tab 602 of tab list 502. The at least one processor may cause interactive fields 604 to appear, through which the user may view and edit or enter data in the indicated categories. The at least one processor may cause header 608 to appear, which shows patient identifying data once the patient information has been saved. The at least one processor may cause the screen 600, and screens under other tabs, to provide a row or arrangement of interactive navigation buttons 606 for performing navigation functions (e.g., previous screen, next screen, save data, cancel data changes) as known in the art. The processor may be configured to save data changes if the user selects “Back” or “Next,” or discard changes, depending on preferences of the user and/or application designer. In an aspect, the processor may require all patient information field to be completed before permitting progress to a subsequent screen (e.g., to the Visit screen or later screens).

FIGS. 6C to 6E depict additional examples of patient information screens. For example, in some embodiments, register patient screen 620 can be displayed when a patient is being registered. The at least one processor may cause interactive fields 624 to appear, through which the user may view and edit or enter data in the indicated categories. According to some embodiments, register patient screen 620 can also include a “Create” button 626 that is configured to create a patient entry in the system, when clicked, based on the data inputted into the interactive fields 624. According to another aspect of some embodiments, once a patient entry has been created (e.g., via “Create” button 626), the at least one processor can cause one or more glucose data authorization buttons (e.g., buttons 628 and 630) to be displayed on register patient screen 620, which may be used to grant and/or request access to a patient's glucose data from the system. While FIG. 6D shows two glucose data authorization buttons 628 and 630 having “Authorize Now” and “Send Auth Link” textual labels, those of skill in the art will understand that other user interface objects, labels, symbols, or pictures can be used in place of (or in addition to) the “Authorize Now” button 628 and the “Send Auth Link” button 630.

According to another aspect of some embodiments, once the system is authorized to access glucose data, the at least one processor can replace the register patient screen 620 with patient information screen 640. In several aspects, patient information screen 640 of FIG. 6E is similar to patient information screen 600 of FIGS. 6A and 6B. For example, interactive fields 604 in FIG. 6A are similar to interactive fields 642 in FIG. 6E. In addition, the at least one processor may cause the screen 640, and screens under other tabs, to provide a row or arrangement of interactive navigation buttons 646 for performing navigation functions (e.g., previous screen, next screen, save data). In some embodiments, navigation buttons 646 does not include a cancel button.

Referring again to FIG. 4, the method 300 may further include, at 420, providing by the at least one processor the information configured for interactive display including visit information enabling entry of test result data in addition to the medical monitoring data. In an aspect of the method 300 illustrated at 430, the visit information enables entry of patient vital data separately from the test result data. In another aspect at 440, the test result data comprises one or more of hemoglobin A1c, total cholesterol, low density lipoproteins, high density lipoproteins, kidney function, and triglycerides. The method may further include, at 450, by the at least one processor, providing prior test result data to the display device for display with test result data entered via the interactive graphical user interface during a most recent session.

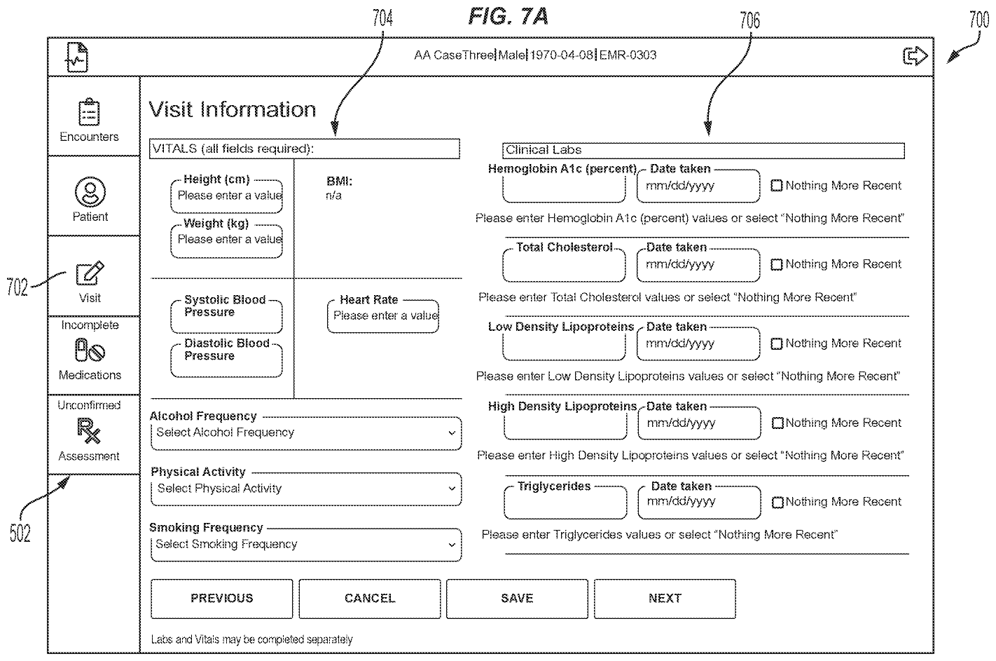

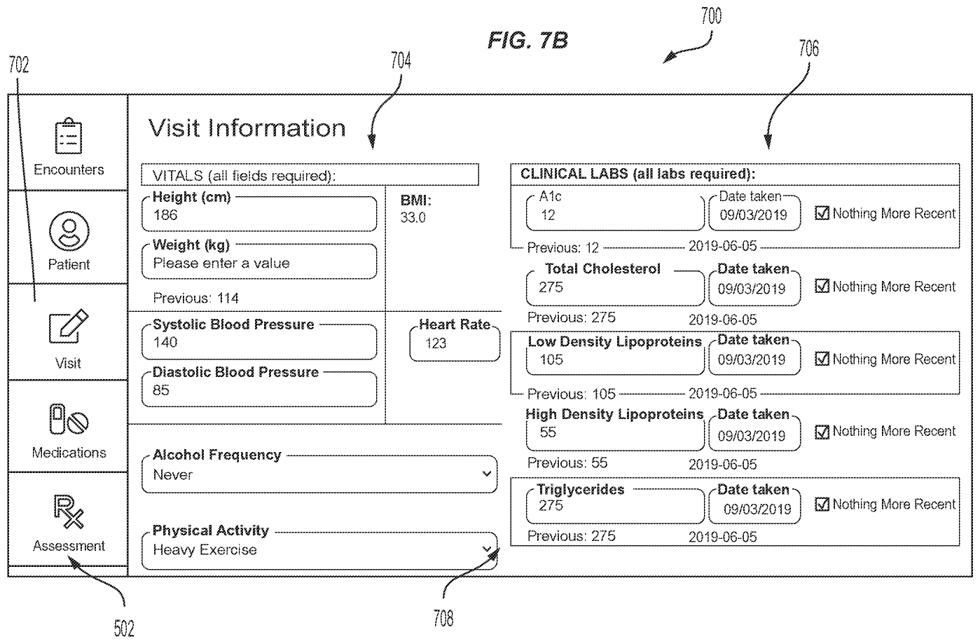

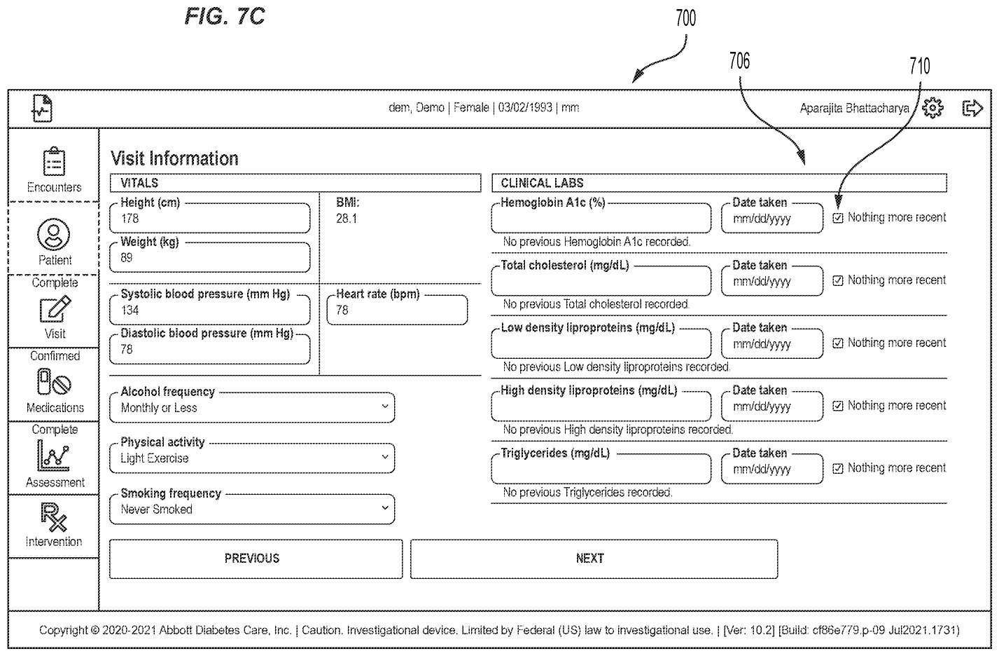

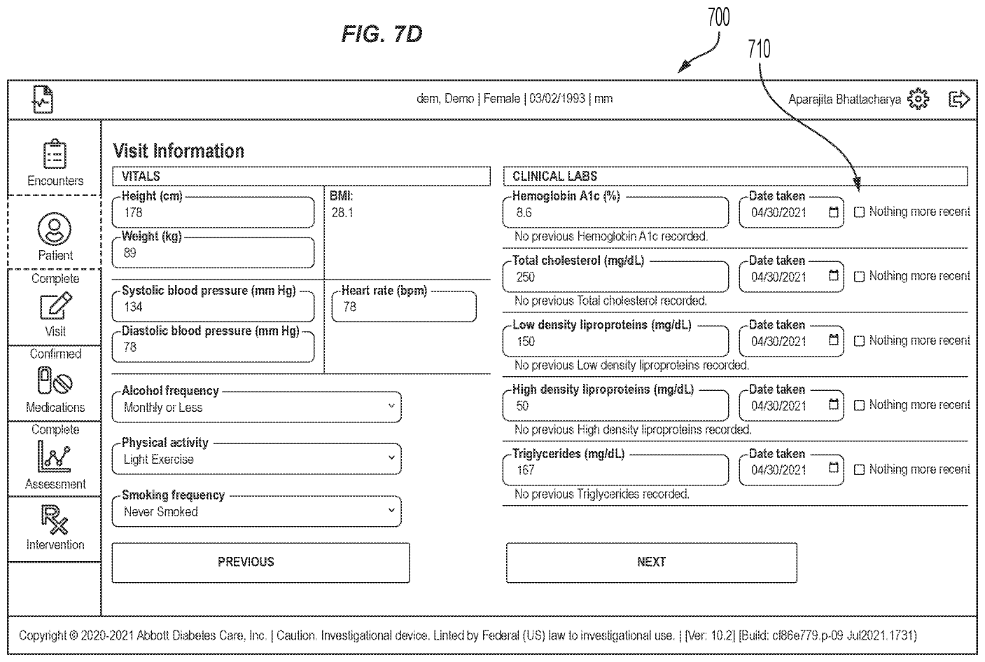

FIGS. 7A and 7B further illustrate the foregoing operations 420-450 by examples of a Visit screen 700 under a highlighted Visit tab 702. As shown in FIG. 7A, the processor may cause a patient identifier to appear in a screen header once the patient data is completely entered. The processor may generate the Visit screen 700 including a section 704 containing fields for entering and viewing patient “vitals” (e.g., height, weight, BMI, blood pressure (e.g., systolic and diastolic blood pressures), heart rate, and habits (e.g., alcohol frequency, physical activity, smoking frequency)) and a section 706 containing fields for entering and viewing laboratory data (e.g., hemoglobin A1c, total cholesterol, low density lipoproteins, high density lipoproteins, and triglycerides) with related measurement dates. In an aspect, the at least one processor may enable to user to complete the sections 704 and 706 separately. As shown in FIG. 7B, the processor may cause the most recent prior values of the lab data to appear on screen 700, as shown at section 708. For example, on subsequent visits, previous values entered for the vitals and lab results may be displayed for reference. Furthermore, according to some embodiments, as depicted in FIGS. 7C and 7D, a “Nothing More Recent” checkbox 710 can be displayed adjacent to each row of data in section 706. According to one aspect of the embodiments, the “Nothing More Recent” checkbox can be selected to indicate that no recent lab measurements are available, such that no values will be entered in the adjacent value and date fields. The at least one processor can display values in the fields as they are entered. If there is no value to be entered, then “Nothing More Recent” checkbox 710 is selected.

The patient data may be manually entered by the health care provider. In addition, or in an alternative, patient data may be automatically retrieved and entered into the interface by any suitable method, for example, via an electronic medical records (EMR) data and communications protocol.

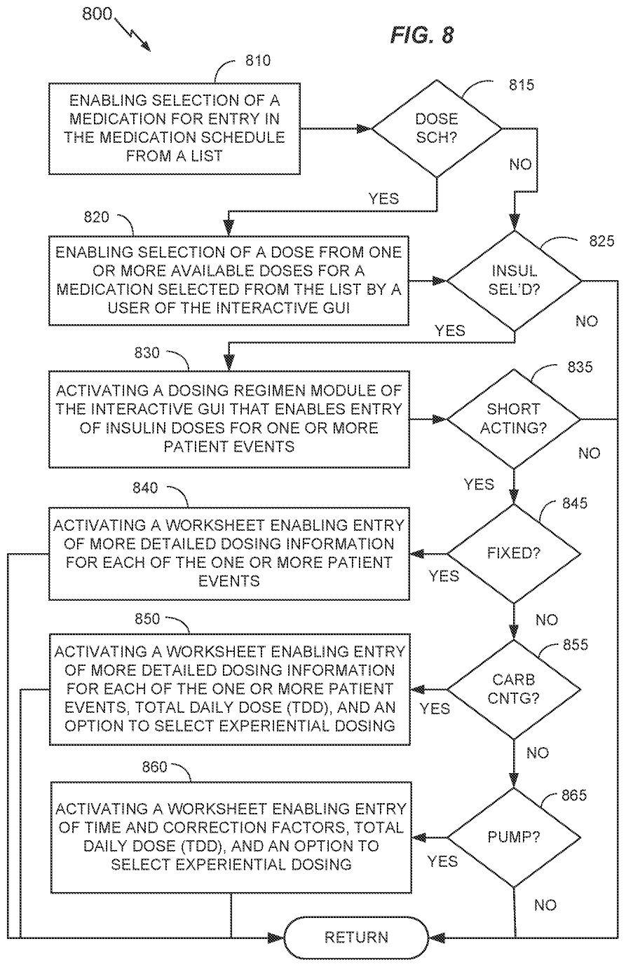

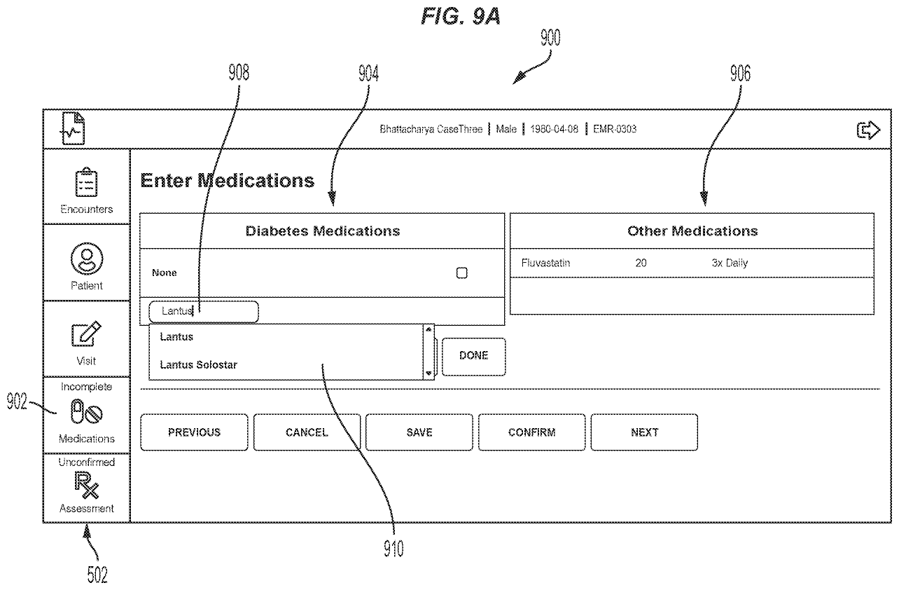

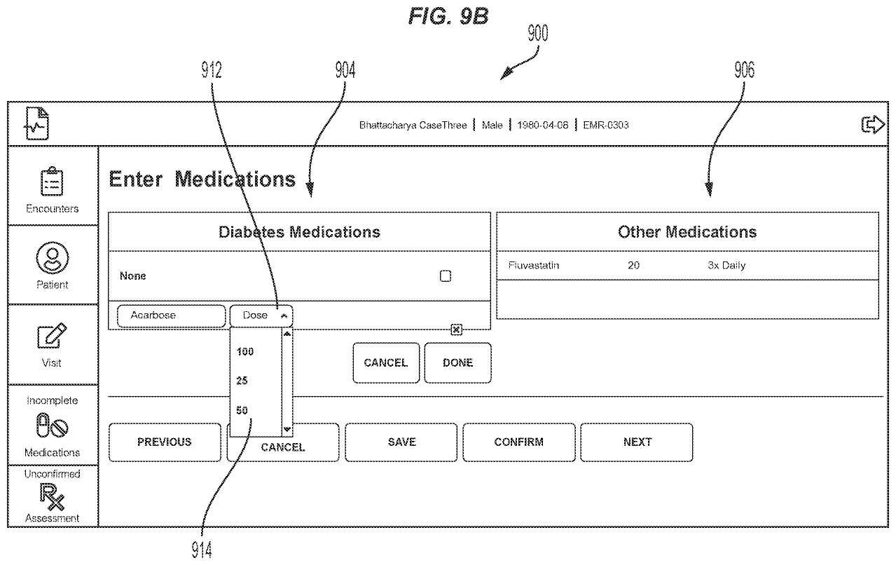

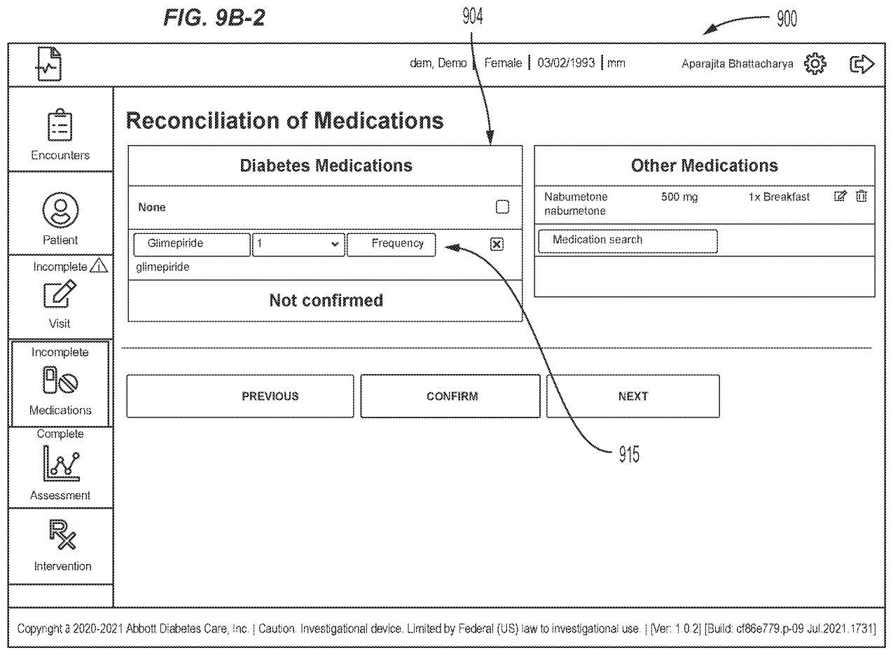

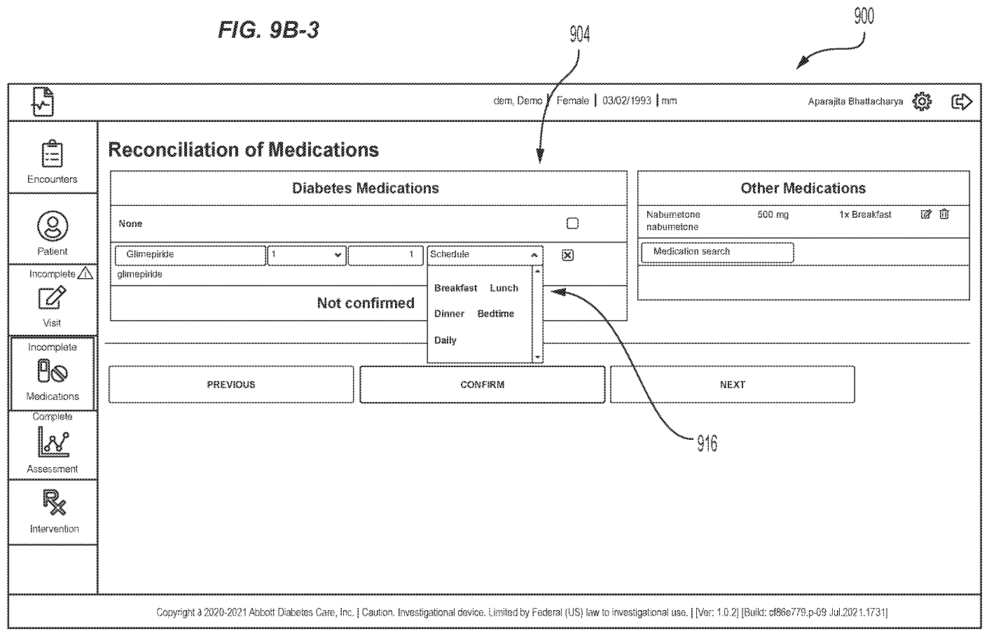

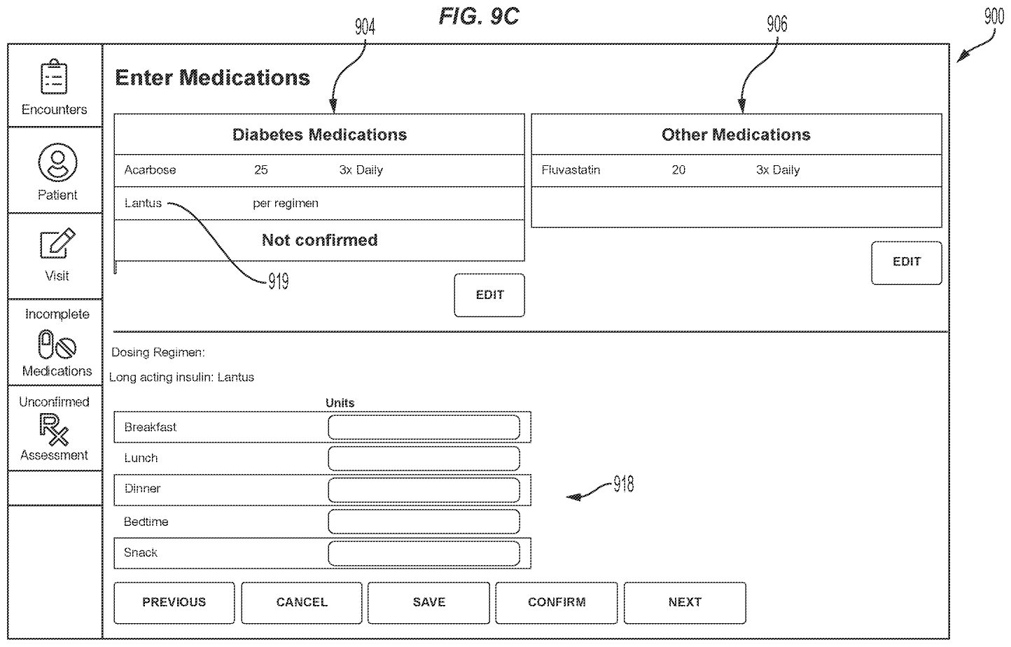

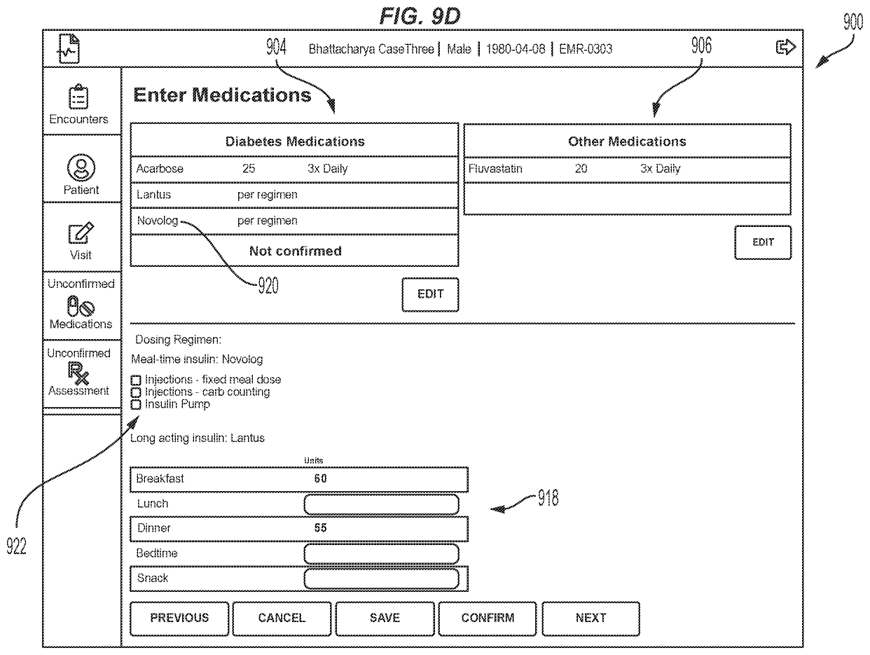

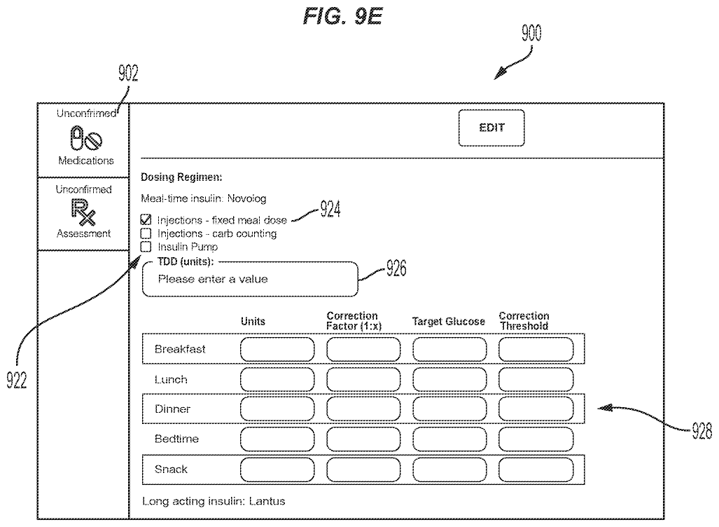

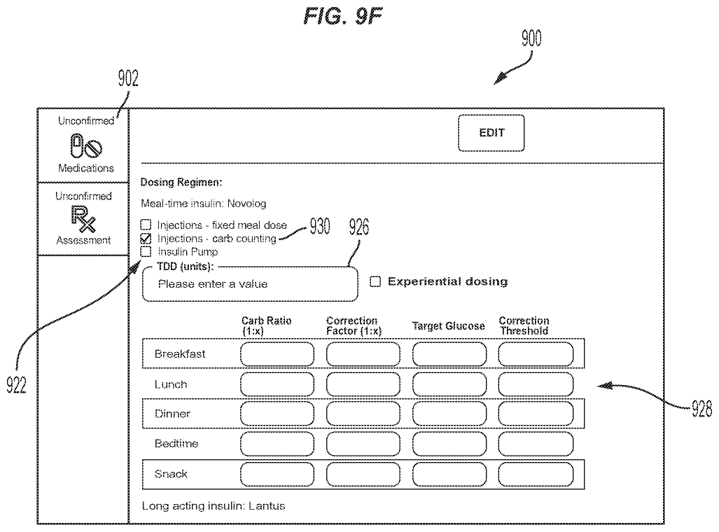

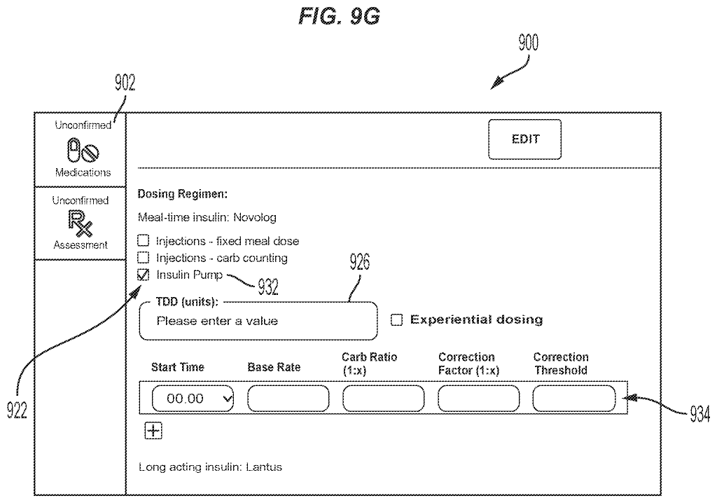

FIG. 8 illustrates further additional operations 800 that may be included as part of the method 300. At 810, the method may include, by the at least one processor, enabling selection of a medication for entry in the medication schedule from a list. FIG. 9A shows an example Medications screen 900 consistent with this operation, wherein the processor causes a list of diabetes medications 904 taken by the patient and a list of other medications 906 to appear. If the user enters characters into the search field 908, the processor may display a selectable list of matching medication names 910. The Medications screen appears under the highlighted medications tab 902 in the tab list 502.

At 820 of FIG. 8, the method 300 may include, by the at least one processor, enabling selection of a dose from one or more available doses for a medication selected from the list by a user of the interactive graphical user interface, is a medication is added for which, at 815, the processor determines a dose schedule is available in its memory. FIG. 9B shows the Medications screen 900 including typical or available dose recommendations for the medication indicated in box 908. The processor response to user selection of a dose from the list 914, which indicates the dose of medication taken by the patient. Additionally, as can be seen in FIGS. 9B-2 and 9B-3, screen 900 can also include a selectable frequency field 915 (e.g., having integer values starting at 1) and/or a selectable schedule field 916 (e.g., Breakfast, Lunch, Dinner, Bedtime, Daily).

At 825 of FIG. 8, if the selected medication is a type of insulin, then at 830 the method may include, by the at least one processor in response to selection of an insulin medication from the list, activating a dosing regimen module of the interactive graphical user interface that enables entry of insulin doses for one or more patient events. FIG. 9C illustrates an example of a Medications screen including a display 918 of dosing regimen, for the insulin medication indicated at 919. For a long-lasting form of insulin such as Lantus, the dosing regimen field in section 918 allows the user to enter a value for insulin dosing at mealtimes and before going to bed.

At 835 of FIG. 8, if the form of insulin is short-acting, the method 300 may include, at 830, the at least one processor enabling the user selection of options only in response to user selection of a short-acting insulin medication from the list. The processor may display a list of options 922 for delivery of the short-acting insulin, as shown in FIG. 9D. These options do not appear when entering a long-lasting form of insulin. In the illustrated example, three options appear: a fixed meal dose administered by injection, a dose based on a carbohydrate count for the meal administered by injection, and a dose administered by insulin pump. At 845, if the short-acting insulin is delivered in a fixed dose, the method 300 may include, at 840, by the at least one processor, in response to user selection of a fixed meal injected dose option from the options for mealtime insulin dosing, activating a worksheet enabling entry of more detailed dosing information for each of the one or more patient events. For example, the worksheet activated at 840 for fixed dose may enable entry of dose amounts, correction factor, target glucose and a correction threshold. In some embodiments, the correction threshold can be automatically populated after a correction factor and a target glucose is inputted, since the correction threshold is equal to the correction factor plus the target threshold (or some other function thereof). FIG. 9E shows a portion of a Medications screen 900 including a more detailed worksheet 928 with additional columns for correction factor, target glucose, and correction threshold, in addition to a field 926 for a Total Daily Dose (TDD) value. The selection of the fixed dose option is indicated at 924.

At 855, if the short-acting insulin is based on carbohydrate counting, the method 300 may further include at 850, by the at least one processor, in response to user selection of a carbohydrate counting injected dose option from the options for mealtime insulin dosing, activating a worksheet enabling entry of more detailed dosing information for each of the one or more patient events, Total Daily Dose (TDD), and an option to select experiential dosing. For example, the carb counting worksheet may enable entry of I:C ratio, correction factor, target glucose and correction threshold. In some embodiments, the correction threshold can be automatically populated after a correction factor and a target glucose is inputted, since the correction threshold is equal to the correction factor plus the target threshold (or some other function thereof). FIG. 9F shows a portion of a Medications screen 900 including the more detailed worksheet 928 with additional columns for correction factor, target glucose, and correction threshold, the field 926 for a Total Daily Dose (TDD) value and the option for experiential dosing. The selection of the carb-counting dose option is indicated at 930.

At 865, if the short-acting insulin is delivered by a pump, the method 300 may further include, at 860, by the at least one processor, in response to user selection of an insulin pump dose option from the options for mealtime insulin dosing, activating a worksheet enabling entry of time, basal rate, I:C ratio, correction threshold and correction factor, Total Daily Dose (TDD), and an option to select experiential dosing. FIG. 9G shows a portion of a Medications screen 900 including the worksheet 934 enabling entry of time and correction factors, the field 926 for a Total Daily Dose (TDD) value and the option for experiential dosing. The selection of the pump delivery option is indicated at 932.

For any of the aforementioned screens, medication data may be manually entered by the health care provider. In addition, or in an alternative, medication data may be automatically retrieved and entered into the interface by any suitable method, for example, via an electronic medical records (EMR) data and communications protocol.

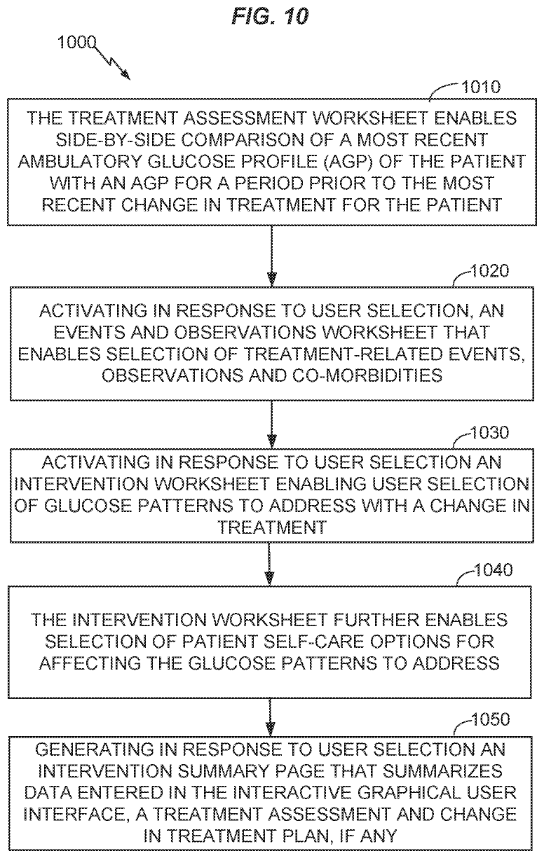

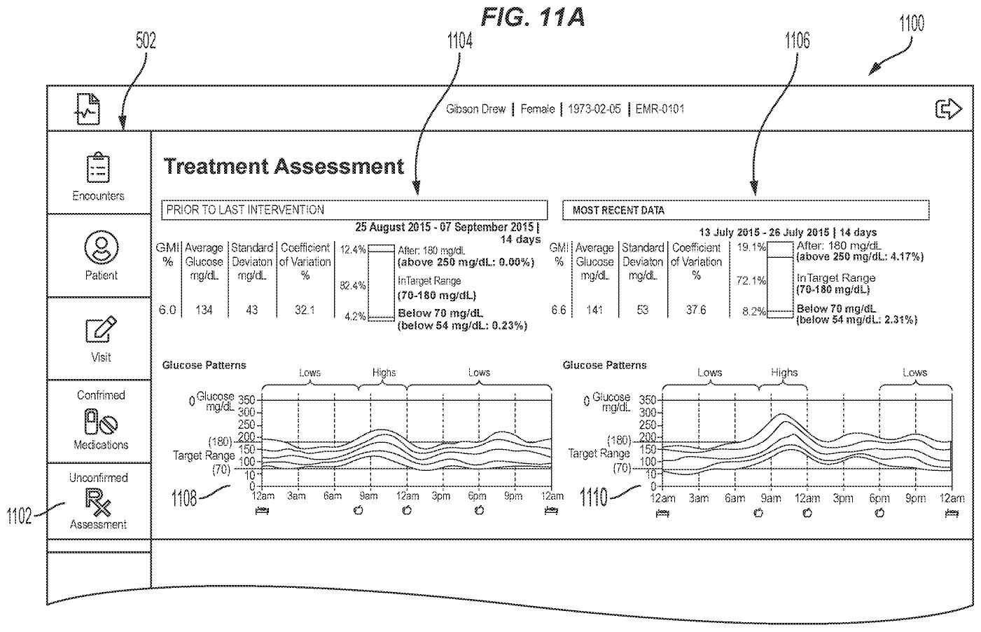

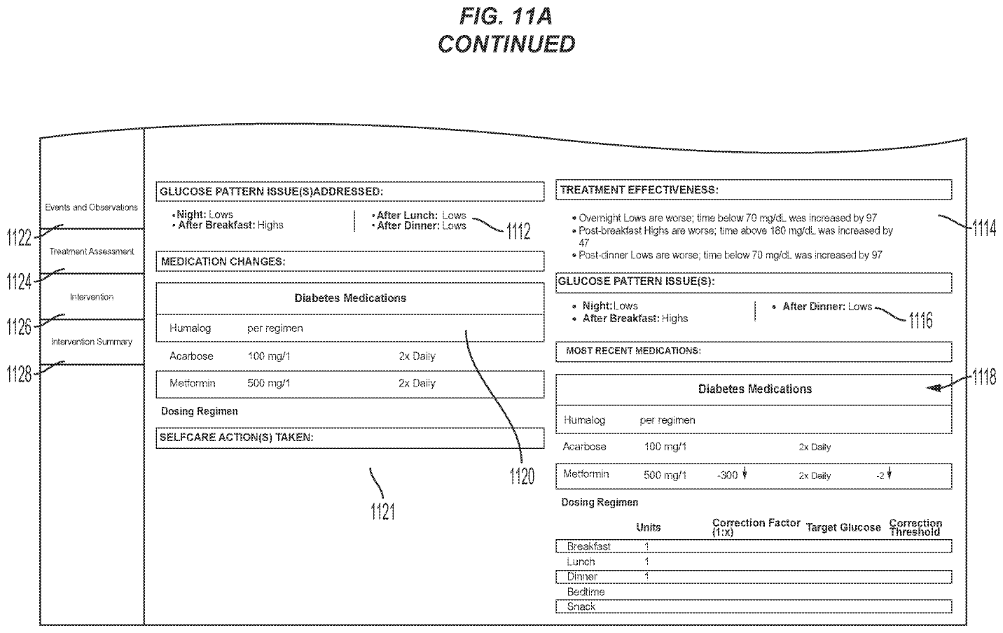

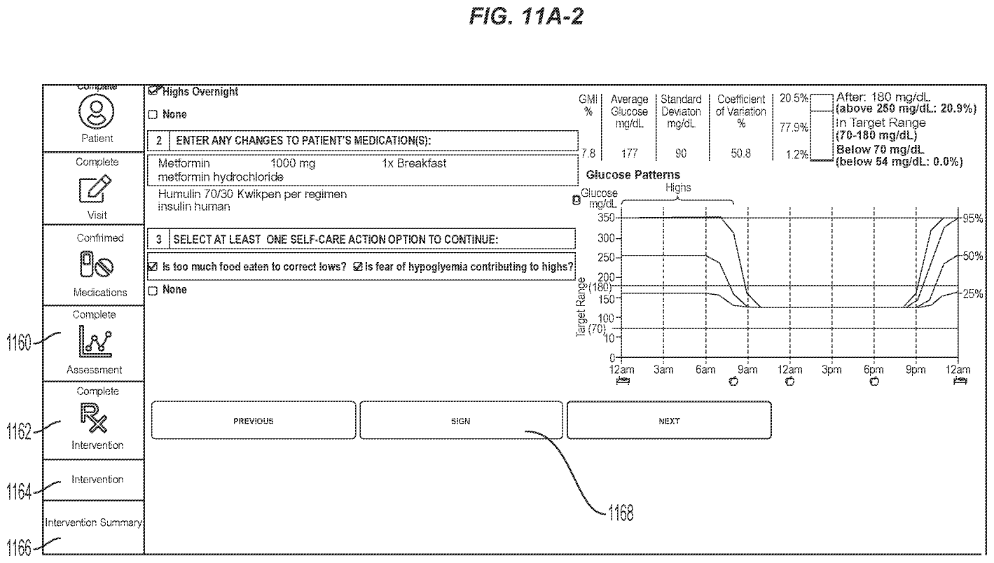

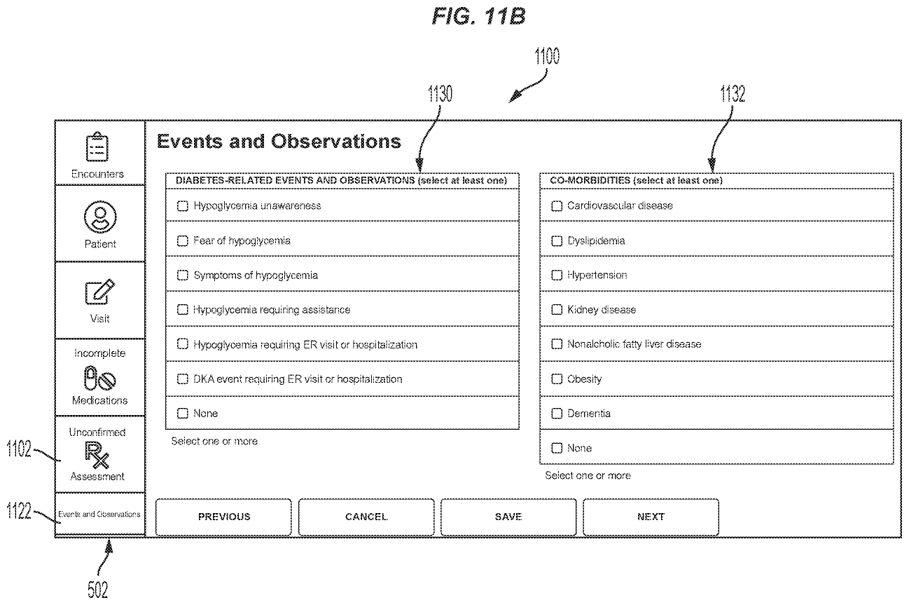

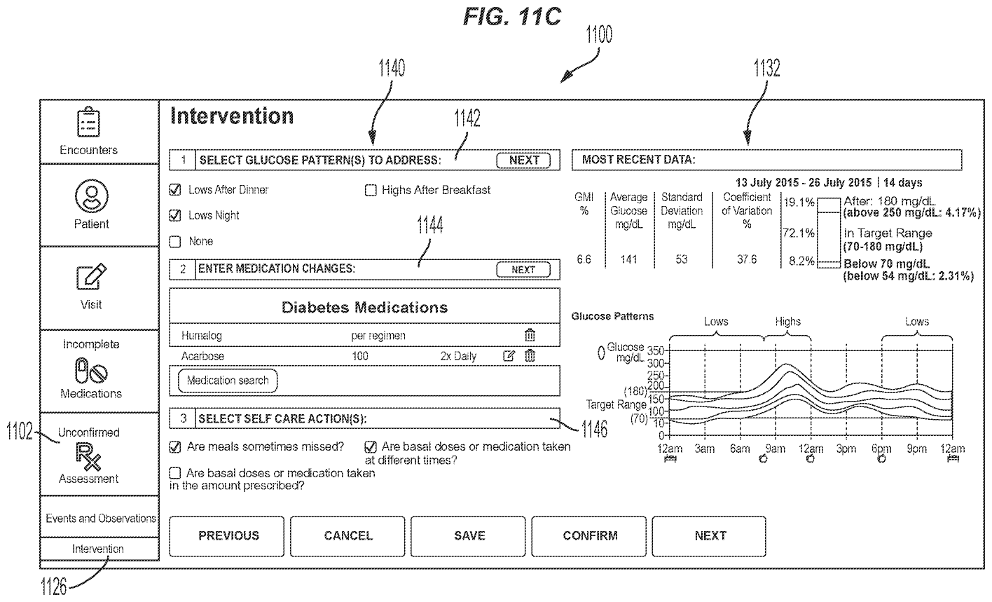

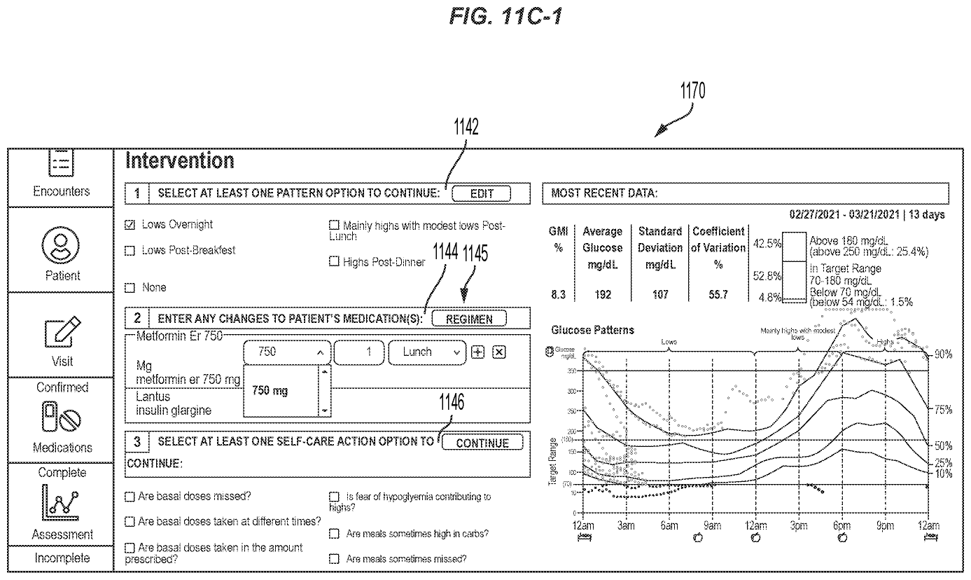

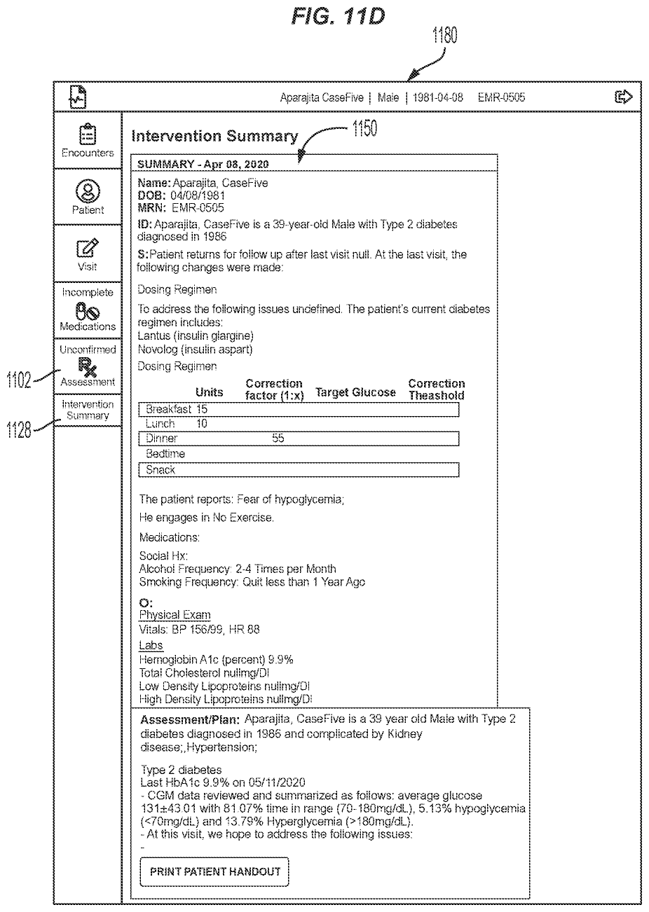

Referring to FIG. 10, the method 300 may include one or more of the additional operations 1000 for treatment assessment. The treatment assessment portion of the interactive graphical user interface enables the user to compare the current AGP with the AGP prior to the last treatment change, review the issues addressed during the prior patient visit, review relative changes in treatment effectiveness, if any, and review changes in medication. At 1010, the method 300 may include the at least one processor preparing the treatment assessment worksheet enabling side-by-side comparison of a most recent Ambulatory Glucose Profile (AGP) of the patient with an AGP for a period prior to the most recent change in treatment for the patient. Side-by-side comparison is illustrated in FIG. 11A, showing a left-most section 1104 displaying monitoring and medication data prior to last intervention, and a right-most section 1106 showing most recent data. The Treatment Assessment screen 1100 appears under an assessment tab 1102, sub-tab 1124 for treatment assessment. Other available sub-tabs may include a sub-tab 1122 for events and observations, a sub-tab 1126 for intervention, and a sub-tab 1128 for intervention summary.

In other embodiments, as depicted in FIG. 11A-2, the assessment 1160 and intervention 1162 sections can comprise separate tabs entirely (i.e., instead of sub-tabs). According to some embodiments, the assessment tab 1160 can include a treatment assessment sub-tab (not shown) and an events and observations sub-tab (not shown). According to another aspect of some embodiments, the intervention tab 1162 can include an intervention sub-tab 1164 and an intervention summary sub-tab 1166, wherein the intervention summary sub-tab 1166 appears only after a visit has been signed off (e.g., by clicking on the sign button 1168).

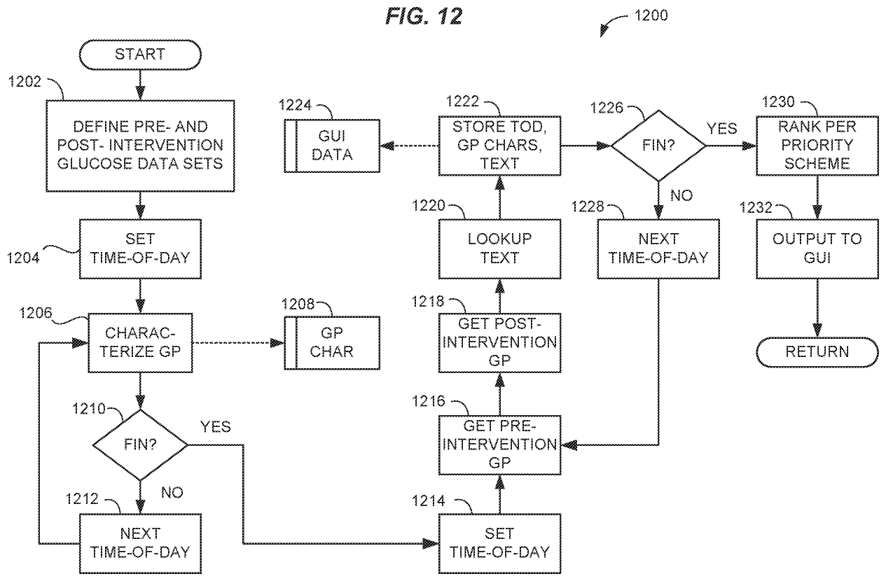

Referring back to FIG. 11A, the treatment assessment data 1104, 1106 may include a comparison of metrics such as GMI, average glucose, standard deviation, and coefficient of variation; a comparison of glucose patterns 1108, 1110 in graph form, a list of glucose patterns addressed during a last visit 1112, a list of current glucose pattern issues 1116, and medication lists 1120, 1118. The section 1120 shows recommended medication changes resulting from the treatment assessment at the patient's last visit. Likewise, the section 1121 may show any recommended self-care changes, if any, identified by the prior treatment assessment. In addition, the most recent data section 1106 may include observations and/or recommendations 1114 regarding treatment effectiveness. These observations and/or recommendations 1114 can be determined using algorithms and logic based on, for example, glucose central tendency (e.g., median) and variability values, and/or time-in-range values (e.g., time below 70 mg/dL, time above 180 mg/dL), many of which are described in further detail in WO 2012/108939, WO 2014/106263, and WO 2014/145335, all of which are herein expressly incorporated by reference in their entirety for all purposes. The algorithms may be run on a cloud-based platform. The glucose pattern issues 1112, 1114 highlighted may include the periods of high or low glucose levels determined to be the most problematic based on the algorithms. Further discussion of an algorithm for providing observations and recommendations are provided herein below in connection with FIG. 12.

Referring to FIG. 10 at 1020, the method 300 can further comprise, by the at least one processor, activating in response to user selection, an events and observations worksheet that enables selection of treatment-related events, observations, and co-morbidities. FIG. 11B shows an example of an assessment screen 1100 under an events and observations sub-tab 1122. The screen 1100 includes a list of diabetes-related events and observations under the left column 1130, and a list of co-morbidities for the patient under the right column 1132. According to some embodiments, the list of co-morbidities and/or their selections, can be carried over from a prior visit. The processor configures the interactive graphical user interface so that each item in both lists 1130, 1132 is selectable by the user.

At 1030, the method 300 may further include by the at least one processor, activating in response to user selection an intervention worksheet enabling user selection of glucose patterns to address with a change in treatment. At 1040, in an aspect the intervention worksheet further enables selection of patient self-care options for affecting the glucose patterns to address. FIG. 11C shows an example of an intervention screen 1170, which, in the depicted embodiment, is under an intervention sub-tab 1126 of the assessment tab 1102. The intervention worksheet 1140 includes a list of user-selectable options 1142 identifying glucose patterns to address, medication changes 1144, and self-care actions 1146. At the right, the processor may display results for most recent data 1132, for convenient reference. After the user completes the intervention screen, the processor may treat the visit as complete and remove the patient from the encounters list.

According to another aspect of some embodiments, certain user interface features can be displayed based on context. For example, in some embodiments, if there are insulin medications already on the list, as described above with respect to FIGS. 9A to 9D, then the medication changes section 1144 can include a “Regimen” button 1145 (instead of a “Next” button), as shown in FIG. 11C-1. The “Regimen” button 1145, when clicked, can be configured to display a medication regimen (e.g., insulin regimen), and allows a user to edit regimen entries. Under other circumstances, medication changes section 1144 can include a button, “Edit,” which allows the medication list to be edited (e.g., medications can be added and/or deleted). Under other circumstances, as depicted in FIG. 11C, the button appears as “Next,” which can direct the user to the self-care actions section 1146.

The processor may determine the glucose patterns as described in U.S. Pat. No. 9,351,670 and/or related applications WO 2012/108939, WO 2014/106263, and WO 2014/145335 referenced above. The patterns are listed in 1142 and may be configured as user selectable links. The intervention screen may be configured to help the user (e.g., a health care practitioner) to identify and prioritize patterns that need to be corrected, either with medication changes or with self-care changes or both. For example, the screen 1170 may include user selectable/enterable fields for medication changes and self-care changes as shown at 1144 and 1146, respectively. The processor associates and store all these user entries with the current visit date. When the same user accesses the system at the next visit, the processor may display the prior patterns addressed, medication changes and self-care changes on the assessment screen, as shown at FIG. 11A, features 1112, 1120, and 1121 described above, to assist the user in assessing how the prior interventions affected the patient's glucose patterns by comparing the data for the current visit with the previous visit when the intervention took place. The assessment screen 1100 may assist the user to assess the effectiveness of prior interventions in the context of recalling which patterns the user was intending to address from the previous visit and/or can also provide information regarding changes in unaddressed patterns as well.

At 1050, the method 300 may further include by the at least one processor, generating in response to user selection an intervention summary page that summarizes data entered in the interactive graphical user interface, a treatment assessment and change in treatment plan, if any. FIG. 11D shows an example of intervention summary 1180, which, in the depicted embodiment, is under a summary sub-tab 1128 of assessment tab 1102. The processor displays a summary 1150 in a standard format suitable for entry in an Electronic Medical Record (EMR). In response to a user selection, the processor may generate the summary in an electronic format and save it in the patient's file as an EMR.

FIG. 12 illustrates one or more additional operations 1200 for providing human-readable observations and recommendations in treatment assessment, sometimes referred to as a Guided Interpretation Report (GIR), that may be included in the method 300, or similar method, for controlling an interactive user interface, for example, a GUI. Observations may include time-of-day patterns, and recommendations may include medication and self-care considerations, which may be prioritized by an algorithm based on a detected relation between glucose patterns for a time-of-day, or for multiple times-of-day. More detailed examples of the operations 1200 and/or related operations may be as described in in WO 2012/108939, WO 2014/106263, and/or WO 2014/145335.

At 1202, the method may further include defining, by the apparatus processor, pre-intervention and post-intervention monitoring datasets for analysis. “Intervention” may refer to a prior visit by the patient to a medical practitioner for which medication and monitoring data as described herein are available and updated by the medical practitioner via the interactive graphical user interface. The post-intervention monitoring dataset may be, or may include, monitoring data collected after the most recent completed intervention. The pre-intervention monitoring data may be, or may include, monitoring data collected prior to the most recent completed intervention and no earlier than the next most recent intervention. In some embodiments, the pre-intervention dataset may include the most recent pre-intervention monitoring data and data from one or more earlier periods. In an aspect, defining the pre-intervention and post-intervention datasets may be performed implicitly based on memory state after the most-recent monitoring data is accessed. In an alternative, or in addition, the apparatus processor may define the datasets based on user input.

At 1204, the processor may set a first time-of-day value or values, based on a predefined time or based on events detectable in the monitoring data. For example, a “post-breakfast” time-of-day may be defined as the period between 8 am and noon, or as the time between signals or data indicating breakfast is eaten until the next meal. At 1206, the processor may analyze the data in the selected time-of-day period to characterize a glucose pattern (GP). The glucose pattern may be a numeric value or values, or may be a text string, for example, “high,” “low,” “moderately high,” moderately low,” “in-range,” etc., or a symbolic indicator thereof. Once determined, the processor may save the GP in a memory 1208 for later use in the operations 1200.

At 1210, the processor checks whether other time-of-day periods exist that are not yet analyzed. At 1212, if another time-of-day period is to be analyzed, the processor defines the next time-of-day period and reverts to block 1206 previously described. The processor repeats the GP characterization loop (1206, 1210, 1212) for the pre-intervention and post-intervention datasets until no further time-of-day periods remain to be analyzed. Then, the processor resets the time-of-day at 1214 to begin an assessment loop for each relevant time-of-day in the data.

At 1216, the processor retrieves a pre-intervention GP and at 1218, retrieves a post-intervention GP, for example, from the memory 1208 where the GPs are stored in relation to the relevant times-of-day. Optionally, the processor may determine or retrieve a secondary factor expressing the influence of a GP for an adjacent time of day. For example, for a post-lunchtime GP, the processor may retrieve a post-breakfast GP as a secondary factor. At 1220, for the current time-of-day, the processor may look up a text value for a comment, observation or recommendation from a table or other data structure, based on the GP for each period, and optionally for one or more secondary factors. For example, it may look up text stored in a record indicated by a row-column intersection wherein the GP for the pre-intervention period indicates the row and the GP for the post-intervention period indicates the column. If secondary factors are used, each factor can be used to define another dimension of the data table. Thus, at 1220 the processor selects text most appropriate for a unique combination of at least a time-of-day period, a pre-intervention GP, and a post-intervention GP. At 1222, the processor may store the unique combination and associated text defined at 1220 in a memory 1224 for later use in the graphical user interface.

At 1226, the processor determines if the assessment loop (1216-1228) is finished. If not, the processor selects the next time-of-day period at 1228 and reverts to block 1216. If so, at 1230, the processor ranks the texts in the memory 1224, or a subset of the texts, in a priority order according to a priority scheme. Any desired priority scheme may be used, for example one based on an associated risk or priority score for each predefined text string or associated unique combination of factors. At 1232, the processor provides the text strings or a high-priority subset thereof, optionally each with its associated factors, for output via the interactive graphical user interface for use in the present intervention. For example, the screenshot 1100 in FIG. 11A shows the selected and prioritized text strings at 1114 and the associated glucose patterns and times-of-day at 1112.

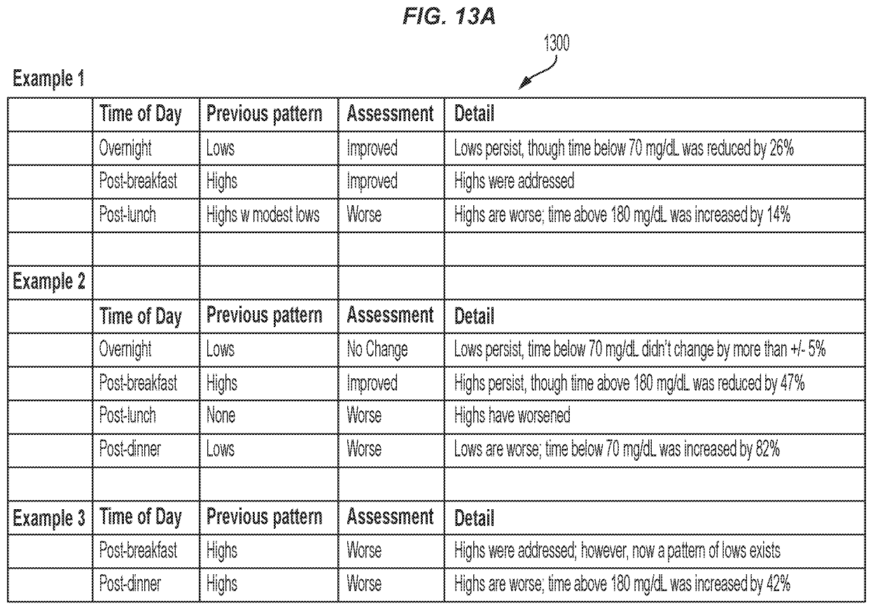

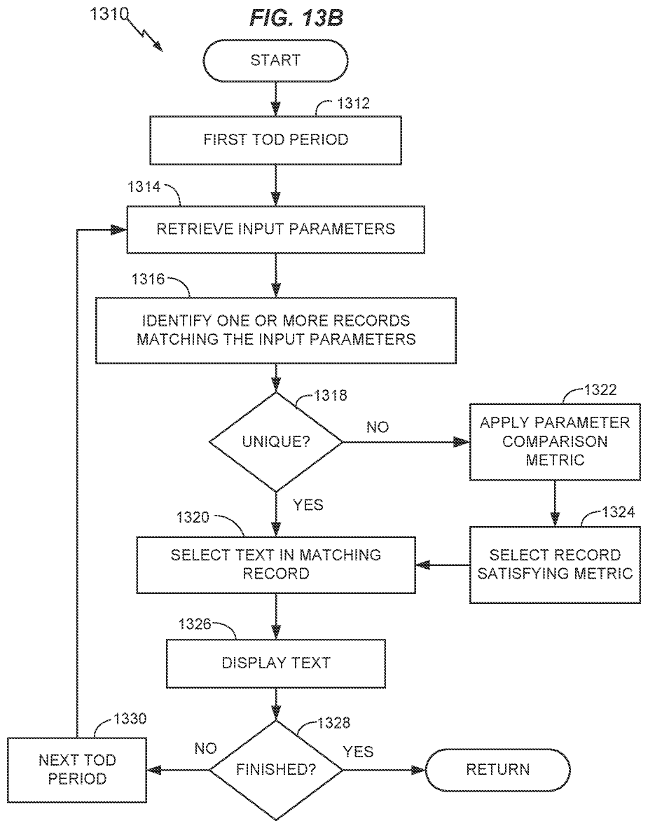

FIG. 13A illustrates examples of patterns and assessments, including assessment details, for diabetes treatment. Table 1300 illustrates some example results of using assessment logic used to determine a recommendation to display to the user. Reading from left to right, the first two columns show examples of input conditions for arriving at a categorical assessment as shown in the third column. More particularly, the first column indicates a time-of-day for a glucose pattern evident detected over a multi-day monitoring period, while the second column shows the pattern observed at the indicated times. The third column shows an assessment of the condition based on comparing a glucose pattern detected for the most recent monitoring period with a glucose pattern for one or more prior periods, for example, for a next-to-most-recent period. The fourth column, titled “Detail,” show examples of predetermined text that may be retrieved from memory as displayed, for example as shown at 1114 of FIG. 11A, based on a selection algorithm as described above.

At least one processor of an apparatus may execute additional operations 1310 as shown in FIG. 13B for performing assessment logic to generate the text as shown in FIG. 13A, for example. The logic may be represented by a data table, or equivalent data structure, that relates the glucose pattern for each time-of-day (TOD) period to the display text via a set of intervening input conditions. The table may be used once for each TOD period (postbreakfast, postlunch, post dinner, overnight). Input columns of the table may be used to identify a row of the table corresponding to a unique set of inputs. For example, a first column may identify a TOD period that the row relates to, a second column may identify a glucose pattern of the previous visit, also called a pre-intervention pattern, a third column may identify a glucose pattern of the current visit (also called a post-intervention pattern), a fourth column may compare the times-in-range (TB70, TA180, etc) of the post-intervention with that of the pre-intervention, and a fifth column identifying the predetermined text.

The additional operations (algorithm) 1310 may include, at 1312, identifying a TOD period for assessment. At 1314, the processor may receive input parameters for the TOD period, including at least a pre-intervention pattern and post-intervention pattern. At 1316, the processor may select one or more records matching the input parameters, for example, the processor may select one or more rows matching an input combination of TOD, pre-intervention pattern, and post-intervention pattern. At 1316, the processor may determine whether the number of records (e.g., rows) selected is greater than one (i.e., whether the record is unique). If the record is unique, at 1320, the processor may select the text for display located in the selected row or record.

For some TOD periods, combinations of the second and third columns may not be unique, i.e., the same combinations may occur for more than one row of the table. In such cases, an additional or “fourth” column may also be used as an input to finally select a unique row. The fourth column may express a relationship between the pre-intervention and post-intervention patterns, for example, a pre-intervention metric less than, greater than, or equivalent to a post-intervention metric. The metric calculations may be based on the glucose data that is used to determine the glucose pattern. Thus, at 1322, the processor may compare this metric for the pre-intervention and post-intervention periods, and at 1324, select a row or record satisfying the comparison metric.

For example, a metric may be, or may include, a percentage of time of glucose below a threshold (e.g., 70 mg/dL). Likewise, for high periods, the metric may be, or may include, a percentage of time the glucose is above a threshold (e.g., 180 mg/dL). The processor may detect “no change” or “equivalent” when both metrics are within a stated range, for example, not greater than 5% difference. Likewise, the processor may apply the threshold when determining “greater than” or “less than,” such that these relations require a difference greater than the threshold.

At 1320, the processor may use a unique combination of the input parameters to select a corresponding unique row containing the output text to be displayed at 1326. Some instances of the text may have a further variable text that is determined by a metric calculation. For example, where {metric text} is indicated, the processor may calculate the difference in the metrics used in the fourth column; for example, the processor may calculate the absolute value of the change in the metric between the current and previous visits. The apparatus may display 1326 the value with percentage units (or time), in line with the rest of the text, as shown in the examples. At 1328, the processor may determine whether additional TOD periods remain to be assessed. If the operations 1310 are not finished, at 1330, the processor may select the next TOD period and revert to block 1314 for the newly selected period. If the method is finished, the processor may revert to the routine that called the assessment logic operations 1310.

In alternative embodiments, the table may include one or more further outputs further categorizing the assessment, for instance to highlight “improvement” assessments by coloring the displayed text green.

In another aspect, the processor may determine an order in which the assessment text is displayed. For example, the table may provide several, for example, up to 4 assessment text sentences, one for each TOD period, for display. The processor may define the order of display by the order indicated in columns. For instance, the processor may display text associated with “low” patterns before that of other patterns. For further example, within a pattern, the processor may display text associated with the TOD starting with the overnight period and arrange subsequent periods in order of their normal daily sequence.

The text examples and assessment logic illustrated and described in connection with FIGS. 13A and 13B are non-limiting. Any suitable text may be developed and saved in memory for selection and display on the graphical user interface, and assessment logic may be adapted for the text and intended use case.

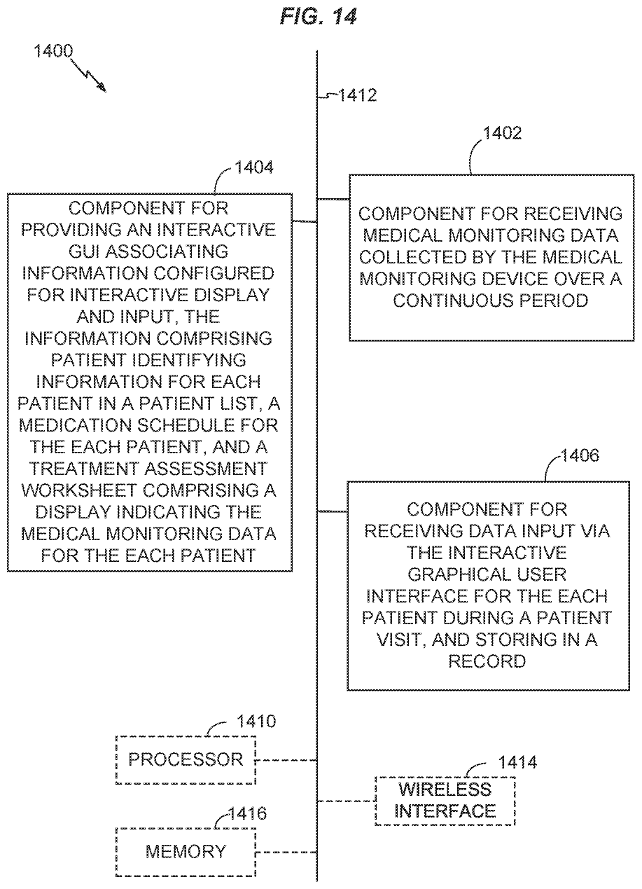

FIG. 14 is a conceptual block diagram illustrating components of an apparatus or system 1400 for providing an interactive graphical user interface as described herein, according to one embodiment. As depicted, the apparatus or system 1400 may include functional blocks that can represent functions implemented by a processor, software, or combination thereof (e.g., firmware).

The apparatus or system 1400 may further comprise an electrical component 1402 for receiving sensor control data collected by the sensor control device over a continuous period. The component 1402 may be, or may include, a means for said receiving. Said means may include the processor 1410 coupled to the memory 1416, and to the wireless interface 1414, the processor executing an algorithm based on program instructions stored in the memory. Such algorithm may include a sequence of more detailed operations, for example, establishing a wireless session with a sensor control device worn by a patient, requesting data from the monitoring device, and receiving monitoring data from the device; or in an alternative, sending an identifier for the patient to a server (not the monitoring device) with a data request, and receiving the monitoring data from the server.

The apparatus or system 1400 may further comprise an electrical component 1404 for providing an interactive graphical user interface associating information configured for interactive display and input, the information comprising patient identifying information for each patient in a patient list, a medication schedule for the each patient, and a treatment assessment worksheet comprising a display indicating the medical monitoring data for the each patient. The component 1404 may be, or may include, a means for said providing. Said means may include the processor 1410 coupled to the memory 1416, and to the input device 1414, the processor executing an algorithm based on program instructions stored in the memory. Such algorithm may include a sequence of more detailed operations, for example, authenticating a user, determining a session state, generating an interactive data object based on the session state, and sending the data object to a graphics processor for rendering and output. In some embodiments, electrical component 1404 can be a standalone component or device. In other embodiments, electrical component 1404 can be an embedded screen in an EMR, for example, such that an HFC can access the electrical component 1404, and one or more of its corresponding functionalities, from within the EMR.

The apparatus or system 1400 may further comprise an electrical component 1406 for receiving data input via the interactive graphical user interface for the each patient during a patient visit, and storing in a record. The component 1406 may be, or may include, a means for said receiving and storing. Said means may include the processor 1410 coupled to the memory 1416, and to the wireless interface 1414, the processor executing an algorithm based on program instructions stored in the memory. Such algorithm may include a sequence of more detailed operations, for example, defining interface objects in relation to variables, activating focus on an interface object in response to user input, associating input from an object in focus to the variable assigned to the object, generating a report of variables received, and sending the report to a computer memory for storage.

The apparatus 1400 may optionally include a processor module 1410 having at least one processor, in the case of the apparatus 1400 configured as a data processor. The processor 1410, in such case, may be in operative communication with the modules 1402-1406 via a bus 1412 or other communication coupling, for example, a network. The processor 1410 may affect initiation and scheduling of the processes or functions performed by electrical components 1402-1406.

In related aspects, the apparatus 1400 may include a wireless interface module 1414 operable for communicating with a sensor control device worn by a patient. In further related aspects, the apparatus 1400 may optionally include a module for storing information, such as, for example, a memory device/module 1416. The computer readable medium or the memory module 1416 may be operatively coupled to the other components of the apparatus 1400 via the bus 1412 or the like. The memory module 1416 may be adapted to store computer readable instructions and data for effecting the processes and behavior of the modules 1402-1406, and subcomponents thereof, or the processor 1410, or the method 300 and one or more of the additional operations 400, 800, 1000, 1200 or 1310 described in connection there with. The memory module 1416 may retain instructions for executing functions associated with the modules 1402-1406. While shown as being external to the memory 1416, it is to be understood that the modules 1402-1406 can exist within the memory 1416.

The various illustrative logical blocks, modules, circuits, and algorithm steps described in connection with the aspects disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the application and design constraints imposed on the overall system. Skilled artisans may implement the described functionality in varying ways for each application, but such implementation decisions should not be interpreted as causing a departure from the scope of the present disclosure.

As used in this application, the terms “component”, “module”, “system”, and the like are intended to refer to a computer-related entity, either hardware, a combination of hardware and software, software, or software in execution. For example, a component may be, but is not limited to being, a process running on a processor, a processor, an object, an executable, a thread of execution, a program, and/or a computer or system of cooperating computers. By way of illustration, both an application running on a server and the server can be a component. One or more components may reside within a process and/or thread of execution and a component may be localized on one computer and/or distributed between two or more computers.

Program instructions may be written in any suitable high-level language, for example, C, C++, C#, JavaScript, or Java™, and compiled to produce machine-language code for execution by the processor. Program instructions may be grouped into functional modules, to facilitate coding efficiency and comprehensibility. It should be appreciated that such modules, even if discernable as divisions or grouping in source code, are not necessarily distinguishable as separate code blocks in machine-level coding. Code bundles directed toward a specific function may be considered to comprise a module, regardless of whether machine code on the bundle can be executed independently of other machine code. In other words, the modules may be high-level modules only.

Various aspects will be presented in terms of systems that may include several components, modules, and the like. It is to be understood and appreciated that the various systems may include additional components, modules, etc. and/or may not include all the components, modules, etc. discussed in connection with the figures. A combination of these approaches may also be used. The various aspects disclosed herein can be performed on electrical devices including devices that utilize touch screen display technologies and/or mouse-and-keyboard type interfaces. Examples of such devices include computers (desktop and mobile), smart phones, personal digital assistants (PDAs), and other electronic devices both wired and wireless.

In addition, the various illustrative logical blocks, modules, and circuits described in connection with the aspects disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. As used herein, a “processor” encompasses any one or functional combination of the foregoing examples.

Operational aspects disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

Furthermore, the one or more versions may be implemented as a method, apparatus, or article of manufacture using standard programming and/or engineering techniques to produce software, firmware, hardware, or any combination thereof to control a computer to implement the disclosed aspects. Non-transitory computer readable media can include but are not limited to magnetic storage devices (e.g., hard disk, floppy disk, magnetic strips . . . ), optical disks (e.g., compact disk (CD), digital versatile disk (DVD), BluRay™ . . . ), smart cards, solid-state devices (SSDs), and flash memory devices (e.g., card, stick). Of course, those skilled in the art will recognize many modifications may be made to this configuration without departing from the scope of the disclosed aspects.

In view of the exemplary systems described supra, methodologies that may be implemented in accordance with the disclosed subject matter have been described with reference to several flow diagrams. While for purposes of simplicity of explanation, the methodologies are shown and described as a series of blocks, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methodologies described herein. Additionally, it should be further appreciated that the methodologies disclosed herein are capable of being stored on an article of manufacture to facilitate transporting and transferring such methodologies to computers.

Non-limiting embodiments are further exemplified in the following numbered clauses:
1. A method for providing human-readable observations and recommendations in treatment assessment for an interactive user interface of a computing device, the method comprising:
   receiving medical monitoring data collected by a sensor control device over a predetermined period;
   defining separate pre-intervention and post-intervention datasets of the medical monitoring data for assessment;
   determining a glucose pattern for each of corresponding time-of-day periods in the separate pre-intervention and post-intervention datasets;
   determining text for display in the interactive user interface based at least in part on the glucose pattern for each of corresponding time-of-day periods; and
   providing the text for output by the interactive user interface.
2. The method of clause 1, wherein the at least one processor determines the text using a data structure to look up a predetermined value indexed by at least two separate indicators of the glucose pattern for each of the corresponding time-of-day periods.
3. The method of clause 1 or 2, wherein the at least one processor further determines the text based on a time-of-day indicator for each of the corresponding time-of-day periods.
4. The method of clause 2 or 3, wherein the at least one processor further determines the text based on at least one additional glucose pattern indicator for at least one period adjacent to the corresponding time-of-day periods.
5. The method of any of clauses 2 to 4, further comprising by the at least one processor providing a signal for displaying the text with the interactive user interface to a display device.
6. The method of any of clauses 1 to 5, wherein defining the pre-intervention and post-intervention datasets is performed implicitly based on memory state after the most-recent monitoring data is accessed.
7. The method of any of clauses 1 to 6, wherein defining the pre-intervention and post-intervention datasets is based on user input via the interactive user interface.
8. The method of any of clauses 1 to 7, further comprising, by the at least one processor analyzing the medical monitoring data, defining the time-of-day periods based on data characteristics indicating one or more meal events.
9. The method of any of clauses 1 to 8, wherein the glucose pattern indicators comprise a high indicator and a low indicator.
10. The method of any of clauses 1 to 9, further comprising, by the at least one processor, ranking texts for different time-of-day periods in a ranked order for the output.
11. The method of any of clauses 1 to 10, further comprising displaying text strings with associated glucose patterns and times-of-day on a user interface device.
12. The method of any of clauses 1 to 11, further comprising processing the pre-intervention dataset to determine a first glucose pattern and separately processing the post-intervention dataset to determine a second glucose pattern.
13. The method of any of clauses 1 to 12, further comprising processing the pre-intervention dataset according to a first processing method and processing the post-intervention dataset according to a second processing method.
14. The method of any of clauses 1 to 13, further comprising automatically defining the pre-intervention and post-intervention datasets based on identifying predetermined patterns in the medical monitoring data.
15. The method of any of clauses 1 to 14, further comprising analyzing the pre-intervention dataset to identify a first glucose pattern or a first glucose event based on a first set of predetermined patterns, and analyzing the post-intervention dataset to identify a second glucose pattern or a second glucose event based on a second set of predetermined patterns.
16. An apparatus for providing human-readable observations and recommendations in treatment assessment for an interactive user interface, comprising at least one processor coupled to a computer memory and to a wireless interface for receiving data from a sensor control device worn by a patient, to memory holding program instructions that when executed by the at least one processor cause the apparatus to perform operations recited by any of clauses 1 to 15.
17. A non-transitory computer-readable medium holding program instructions that when executed by a processor cause an apparatus to perform operations recited by any of clauses 1 to 15.
18. An apparatus comprising means for performing operations recited by any of clauses 1 to 15.

In summary, an interactive graphical user interface associates information configured for interactive display and input, including patient identifying information for each patient in a patient list, a medication schedule for the each patient, and a treatment assessment worksheet comprising a display indicating the medical monitoring data for the each patient. The worksheet enables comparing monitoring results over different periods of time and development of treatment plans.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. Thus, the foregoing description of specific embodiments of the disclosed subject matter has been presented for purposes of illustration and description. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents. Furthermore, any features, functions, steps, or elements of the embodiments may be recited in or added to the claims, as well as negative limitations that define the inventive scope of the claims by features, functions, steps, or elements that are not within that scope.

1-47. (canceled)

48. A system for providing human-readable observations and recommendations in treatment assessment for an interactive user interface, the system comprising:
a wireless communication circuitry configured to receive medical monitoring data from a sensor control device worn by a patient;
at least one processor coupled with the wireless communication circuitry and a memory, the memory storing program instructions that, when executed by the at least one processor, cause the at least one processor to:
define separate pre-intervention and post-intervention datasets of the received medical monitoring data;
determine a glucose pattern for each of corresponding time-of-day periods in the separate pre-intervention and post-intervention datasets;
determine text for display in the interactive user interface based at least in part on the glucose pattern for each of corresponding time-of-day periods; and
output the text to the interactive user interface.

49. The system of claim 48, wherein the program instructions, when executed by the at least one processor, cause the at least one processor to determine the text for display in the user interface using a data structure to look up a predetermined value indexed by at least two separate indicators of the glucose pattern for each of the corresponding time-of-day periods.

50. The system of claim 48, wherein the program instructions, when executed by the at least one processor, cause the at least one processor to determine the text for display in the user interface based on a time-of-day indicator for each of the corresponding time-of-day periods.

51. The system of claim 49, wherein the program instructions, when executed by the at least one processor, cause the at least one processor to determine the text for display in the user interface based on at least one additional glucose pattern indicator for at least one period adjacent to the corresponding time-of-day periods.

52. The system of claim 49, wherein the program instructions, when executed by the at least one processor, further cause the at least one processor to provide a signal for displaying the text to the interactive user interface.

53. The system of claim 48, wherein defining the pre-intervention and post-intervention datasets is performed implicitly based on memory state after the most-recent monitoring data is accessed.

54. The system of claim 48, wherein defining the pre-intervention and post-intervention datasets is based on user input via the interactive user interface.

55. The system of claim 48, wherein the program instructions, when executed by the at least one processor, further cause the at least one processor to:
analyze medical monitoring data collected by a sensor control device over a predetermined period, and
define the time-of-day periods based on data characteristics indicating one or more meal events.

56. The system of claim 48, wherein the glucose pattern indicators comprise a high indicator and a low indicator.

57. The system of claim 48, wherein the program instructions, when executed by the at least one processor, further cause the at least one processor to rank texts for different time-of-day periods in a ranked order for the output.

58. The system of claim 48, wherein the program instructions, when executed by the at least one processor, further cause the apparatus to display text strings with associated glucose patterns and times-of-day on a user interface device.

59. The system of claim 48, wherein the program instructions, when executed by the at least one processor, further cause the at least one processor to process the pre-intervention dataset to determine a first glucose pattern and separately process the post-intervention dataset to determine a second glucose pattern.

60. The system of claim 48, wherein the program instructions, when executed by the at least one processor, further cause the at least one processor to process the pre-intervention dataset according to a first processing method and process the post-intervention dataset according to a second processing method.

61. The system of claim 48, wherein the program instructions, when executed by the at least one processor, further cause the at least one processor to automatically define the pre-intervention and post-intervention datasets based on identifying predetermined patterns in the medical monitoring data.

62. The system of claim 48, wherein the program instructions, when executed by the at least one processor, further cause the at least one processor to:
analyze the pre-intervention dataset to identify a first glucose pattern or a first glucose event based on a first set of predetermined patterns, and
analyze the post-intervention dataset to identify a second glucose pattern or a second glucose event based on a second set of predetermined patterns.
